(19) United States
(12) Patent Application Publication
(10) Pub. No.: US 2024/0344080 A1
(43) Pub. Date: Oct. 17, 2024

(54) METHODS OF IDENTIFYING, SELECTING, AND PRODUCING ANTHRACNOSE STALK ROT RESISTANT CROPS

(71) Applicant: PIONEER HI-BRED INTERNATIONAL, INC., JOHNSTON, IA (US)

(72) Inventors: ALYSSA MARIE DELEON, DES MOINES, IA (US); KEVIN A. FENGLER, CLIVE, IA (US); MARK TIMOTHY JUNG, URBANDALE, IA (US); GIRMA M. TABOR, JOHNSTON, IA (US); SHAWN THATCHER, URBANDALE, IA (US); PETRA J. WOLTERS, KENNETT SQUARE, PA (US)

(73) Assignee: PIONEER HI-BRED INTERNATIONAL, INC., JOHNSTON, IA (US)

(21) Appl. No.: 18/684,373

(22) PCT Filed: Jun. 8, 2022
(86) PCT No.: PCT/US22/72826

§ 371 Date: Feb. 16, 2024

Related U.S. Application Data
(63) Continuation of application No. PCT/US2021/046227, filed on Aug. 17, 2021.

(51) Int. Cl. C12N 15/82; C12Q 1/6874; C12Q 1/6895

(52) U.S. Cl. CPC C12N 15/8282; C12N 15/8213; C12Q 1/6874; C12Q 1/6895; C12Q 2600/13

(57) ABSTRACT

The field is related to plant breeding and methods of identifying and selecting plants with resistance to Anthracnose stalk rot. Provided are methods to identify novel genes that encode proteins providing plant resistance to Anthracnose stalk rot and uses thereof. These disease resistant genes are useful in the production of resistant plants through breeding, transgenic modification, or genome editing.

### FIELD

The field is related to plant breeding and methods of identifying and selecting plants with resistance to Anthracnose stalk rot. Provided are methods to identify novel genes that encode proteins providing plant resistance to Anthracnose stalk rot and uses thereof. These disease resistant genes are useful in the production of resistant plants through breeding, transgenic modification, or genome editing.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 8931WOPCT SEQ LISTING_ST25.txt, a creation date of May 31, 2022, and a size of 34 KB. The sequence listing filed via EFS-Web is part of the specification and is hereby incorporated in its entirety by reference herein.

### BACKGROUND

Anthracnose stalk rot (ANTROT) caused by the fungal pathogen Colletotrichum graminicola (Ces.) Wils, (Cg) is one of the major stalk rot diseases in maize (Zea mays L.). ANTROT is a major concern due to significant reduction in yield, grain weight and quality. Yield losses occur from premature plant death that interrupts filling of the grain and from stalk breakage and lodging that causes ears to be lost in the field. ANTROT occurs in all corn growing areas and can result in 10 to 20% losses.

Farmers can combat infection by fungi such as anthracnose through the use of fungicides, but these have environmental side effects and require monitoring of fields and diagnostic techniques to determine which fungus is causing the infection so that the correct fungicide can be used. The use of corn lines that carry genetic or transgenic sources of resistance is more practical if the genes responsible for resistance can be incorporated into elite, high yielding germplasm without reducing yield. Genetic sources of resistance to Cg have been described (White, et al. (1979) Annu. Corn Sorghum Res. Conf. Proc. 34:1-15; Carson. 1981. Sources of inheritance of resistance to anthracnose stalk rot of corn. Ph.D. Thesis, University of Illinois, Urbana-Champaign; Badu-Apraku et al., (1987) Phytopathology 77:957-959; Toman et al. 1993. Phytopathology, 83:981-986; Cowen, N et al. (1991) Maize Genetics Conference Abstracts 33; Jung, et al., 1994. Theoretical and Applied Genetics, 89:413-418). However, introgression of resistance can be highly complex.

Selection through the use of molecular markers associated with the anthracnose stalk rot resistance trait allows selections based solely on the genetic composition of the progeny. As a result, plant breeding can occur more rapidly, thereby generating commercially acceptable maize plants with a higher level of resistance to anthracnose stalk rot. There are multiple QTL controlling resistance to anthracnose stalk rot (e.g. rcg1 and rcg1b on chromosome 4 (WO2008157432 and WO2006107931)), with each having a different effect on the trait. Thus, it is desirable to provide compositions and methods for identifying and selecting maize plants with newly conferred or enhanced anthracnose stalk rot resistance. These plants can be used in breeding programs to generate high-yielding hybrids that are resistant to anthracnose stalk rot. There is a continuous need for disease-resistant plants and methods to find disease resistant genes.

### SUMMARY

Methods and compositions are provided for identifying and selecting plants with resistance to Anthracnose stalk rot.

In one aspect, provided herein is a method of obtaining a progeny maize plant comprising a marker allele associated with anthracnose stalk rot resistance. The method comprises: a. providing a population of maize plants and isolating nucleic acids from each of the population of maize plants; b. analyzing each of the isolated nucleic acids for the presence of a marker allele on chromosome 10 that is associated with anthracnose stalk rot resistance, wherein said marker allele comprises: i. a “C” at PHM12 at position 61 of SEQ ID NO: 21, ii. a “T” at 19705-9 at position 56 of SEQ ID NO: 22, iii. an “A” at 19707-15 at position 51 of SEQ ID NO: 23, iv. a “G” at C01964-1 at position 201 of SEQ ID NO: 15, v. a “T” at C01957-1 at position 201 of SEQ ID NO: 16, vi. an “A” at SBD_INBREDA_24 at position 51 of SEQ ID NO: 24, vii. an “A” at PHM10 at position 51 of SEQ ID NO: 25, and viii. an “A” at SBD_INBREDA_109 at position 51 of SEQ ID NO: 26; c. selecting one or more maize plant in which said marker allele is detected; d. crossing the selected one or more maize plants with one or more second maize plants to obtain a progeny plant comprising said marker allele.

In a second aspect, provided herein is a method of identifying a plant with an NLR04 allele associated with increased resistance to Anthracnose stalk rot, said method comprising: a. obtaining nucleic acid sample from a maize plant, plant cell, or germplasm thereof; and b. screening the sample for a sequence comprising: (i). a polynucleotide encoding; a polypeptide having the amino acid sequence set forth in SEQ ID NO: 30; (ii). a polynucleotide comprising a sequence set forth in SEQ ID NO: 28; or (iii). one or more marker alleles within 5 cM of (i) or (ii) that are linked to and associated with (i) or (ii). For example, the method can include screening the sample for a marker allele that comprises: i. a “C” at PHM12 at position 61 of SEQ ID NO: 21, ii. a “T” at 19705-9 at position 56 of SEQ ID NO: 22, iii. an “A” at 19707-15 at position 51 of SEQ ID NO: 23, iv. a “G” at C01964-1 at position 201 of SEQ ID NO: 15, v. a “T” at C01957-1 at position 201 of SEQ ID NO: 16, vi. an “A” at SBD_INBREDA_24 at position 51 of SEQ ID NO: 24, vii. an “A” at PHM10 at position 51 of SEQ ID NO: 25, and viii. an “A” at SBD_INBREDA_109 at position 51 of SEQ ID NO: 26.

In another aspect, provided herein is a method of increasing resistance to Anthracnose stalk rot in a plant, comprising expressing in a plant a heterologous polynucleotide that encodes a polypeptide having an amino acid sequence of at least 90% sequence identity to an amino acid sequence of SEQ ID NO: 30; wherein the plant expressing the heterologous polypeptide has increased resistance to Anthracnose stalk rot in the plant when compared to a control plant not comprising the heterologous polynucleotide. In one example, the heterologous polynucleotide is operably linked to a heterologous promoter.

In yet another aspect, provided herein is a method of identifying an allelic variant of the NLR 04 gene, wherein said allelic variant is associated with increased tolerance to Anthracnose stalk rot, the method comprising the steps of: (a). obtaining a population of plants, wherein said plants exhibit differing levels of Anthracnose stalk rot resistance; (b). evaluating allelic variations with respect to the polynucleotide sequence encoding a protein comprising SEQ ID NO: 30, or in the genomic region that regulates the expression of the polynucleotide encoding the protein; (c). associating allelic variations with increased resistance to Anthracnose stalk rot; and (d). identifying an allelic variant that is associated with increased resistance to Anthracnose stalk rot.

In another aspect, provided herein is a method of introducing an allelic variant of a NLR 04 gene, the method comprising introducing a mutation in the endogenous NLR 04 gene such that the allelic variant comprises a polynucleotide sequence encoding a protein that is at least 90% identical to SEQ ID NO: 30 and said allelic variant is associated with increased resistance to Anthracnose stalk rot, wherein the mutation is introduced using zinc finger nuclease, Transcription Activator-like Effector Nuclease (TALEN), the CRISPR/Cas system, or meganuclease.

Also provided herein is a recombinant DNA construct comprising a polynucleotide operably linked to at least one regulatory sequence wherein said polynucleotide comprises a nucleic acid sequence encoding an amino acid sequence of at least 90%, 95%, 96%, 97%, 98%, 99%, 100% sequence identity to SEQ ID NO: 30 and wherein said allelic variant is associated with increased resistance to Anthracnose stalk rot. For example, the regulatory sequence can be a promoter functional in a plant cell. Further provided is a transgenic plant, plant cell or seed thereof comprising the foregoing recombinant DNA construct.

### BRIEF DESCRIPTION OF THE SEQUENCE LISTING

### Description of the Sequence Listing

| SEQ ID NO | Sequence Description |
|---|---|
| 1 | PHM12565-12 |
| 2 | PHM1822-25 |
| 3 | PHM2828-83 |
| 4 | PHM13727-29 |
| 5 | PHM3922-31 |
| 6 | PHM15854-7 |
| 7 | PHM9369-27 |
| 8 | PHM11874-27 |
| 9 | PHM14796-110 |
| 10 | SYN17615 |
| 11 | SYN17616 |
| 12 | C00429-801 |
| 13 | SYN17612 |
| 14 | SYN12395 |
| 15 | C01964-1 |
| 16 | C01957-1 |
| 17 | SYN17244 |
| 18 | SYN14840 |
| 19 | SYN17783 |
| 20 | PZE-110006361 |
| 21 | PHM12 |
| 22 | 19705-9 |
| 23 | 19707-15 |
| 24 | SBD_INBREDA_24 |
| 25 | PHM10 |
| 26 | SBD_INBREDA_109 |
| 27 | NLR 04.AA PRO |
| 28 | NLR 04.AA |
| 29 | NLR 04.AA TERM |
| 30 | NLR 04 PROT |

### DETAILED DESCRIPTION

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof, and so forth. All technical and scientific terms used herein have the same meaning as commonly understood to one of ordinary skill in the art to which this disclosure belongs unless clearly indicated otherwise.

The NBS-LRR (“NLR”) group of R-genes is the largest class of R-genes discovered to date. In Arabidopsis thaliana, over 150 are predicted to be present in the genome (Meyers, et al., (2003), Plant Cell, 15:809-834; Monosi, et al., (2004), Theoretical and Applied Genetics, 109:1434-1447), while in rice, approximately 500 NLR genes have been predicted (Monosi, (2004) supra). The NBS-LRR class of R genes is comprised of two subclasses. Class 1 NLR genes contain a TIR-Toll/Interleukin-1 like domain at their N′ terminus; which to date have only been found in dicots (Meyers, (2003) supra; Monosi, (2004) supra). The second class of NBS-LRR contain either a coiled-coil domain or an (nt) domain at their N terminus (Bai, et al. (2002) Genome Research, 12:1871-1884; Monosi, (2004) supra; Pan, et al., (2000), Journal of Molecular Evolution, 50:203-213). Class 2 NBS-LRR have been found in both dicot and monocot species. (Bai, (2002) supra; Meyers, (2003) supra; Monosi, (2004) supra; Pan, (2000) supra).

The NBS domain of the gene appears to have a role in signaling in plant defense mechanisms (van der Biezen, et al., (1998), Current Biology: CB, 8:R226-R227). The LRR region appears to be the region that interacts with the pathogen AVR products (Michelmore, et al., (1998), Genome Res., 8:1113-1130; Meyers, (2003) supra). This LRR region in comparison with the NB-ARC (NBS) domain is under a much greater selection pressure to diversify (Michelmore, (1998) supra; Meyers, (2003) supra; Palomino, et al., (2002), Genome Research, 12:1305-1315). LRR domains are found in other contexts as well; these 20-29-residue motifs are present in tandem arrays in a number of proteins with diverse functions, such as hormone—receptor interactions, enzyme inhibition, cell adhesion and cellular trafficking. A number of recent studies revealed the involvement of LRR proteins in early mammalian development, neural development, cell polarization, regulation of gene expression and apoptosis signaling. An allele is “associated with” a trait when it is part of or linked to a DNA sequence or allele that affects the expression of the trait. The presence of the allele is an indicator of how the trait will be expressed.

As used to herein, “disease resistant” or “have resistance to a disease” refers to a plant showing increase resistance to a disease compared to a control plant. Disease resistance may manifest in fewer and/or smaller lesions, increased plant health, increased yield, increased root mass, increased plant vigor, less or no discoloration, increased growth, reduced necrotic area, or reduced wilting. In some embodiments, an allele may show resistance one or more diseases.

A plant having disease resistance may have 5, 10, 15, 20, 25, 30, 35, 40, 45, 50, 55, 60, 65, 70, 75, 80, 85, 90, 95, or 100% increased resistance to a disease compared to a control plant. In some embodiments, a plant may have 5, 10, 15, 20, 25, 30, 35, 40, 45, 50, 55, 60, 65, 70, 75, 80, 85, 90, 95, or 100% increased plant health in the presence of a disease compared to a control plant. In some embodiments, a plant comprising

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The phrase “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to ANTROT). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

The term “crossed” or “cross” refers to a sexual cross and involved the fusion of two haploid gametes via pollination to produce diploid progeny (e.g., cells, seeds or plants). The term encompasses both the pollination of one plant by another and selfing (or self-pollination, e.g., when the pollen and ovule are from the same plant).

An “elite line” is any line that has resulted from breeding and selection for superior agronomic performance.

An “exotic strain,” a “tropical line,” or an “exotic germplasm” is a strain derived from a plant not belonging to an available elite line or strain of germplasm. In the context of a cross between two plants or strains of germplasm, an exotic germplasm is not closely related by descent to the elite germplasm with which it is crossed. Most commonly, the exotic germplasm is not derived from any known elite line, but rather is selected to introduce novel genetic elements (typically novel alleles) into a breeding program.

A “favorable allele” is the allele at a particular locus (a marker, a QTL, a gene etc.) that confers, or contributes to, an agronomically desirable phenotype, e.g., disease resistance, and that allows the identification of plants with that agronomically desirable phenotype. A favorable allele of a marker is a marker allele that segregates with the favorable phenotype.

“Genetic markers” are nucleic acids that are polymorphic in a population and where the alleles of which can be detected and distinguished by one or more analytic methods, e.g., RFLP, AFLP, isozyme, SNP, SSR, and the like. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

“Germplasm” refers to genetic material of or from an individual (e.g., a plant), a group of individuals (e.g., a plant line, variety or family), or a clone derived from a line, variety, species, or culture, or more generally, all individuals within a species or for several species (e.g., maize germplasm collection or Andean germplasm collection). The germplasm can be part of an organism, cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells, that can be cultured into a whole plant.

A “haplotype” is the genotype of an individual at a plurality of genetic loci, i.e. a combination of alleles. Typically, the genetic loci described by a haplotype are physically and genetically linked, i.e., on the same chromosome segment.

The term “heterogeneity” is used to indicate that individuals within the group differ in genotype at one or more specific loci.

The heterotic response of material, or “heterosis”, can be defined by performance which exceeds the average of the parents (or high parent) when crossed to other dissimilar or unrelated groups.

A “heterotic group” comprises a set of genotypes that perform well when crossed with genotypes from a different heterotic group (Hallauer et al. (1998) Corn breeding, p. 463-564. In G. F. Sprague and J. W. Dudley (ed.) Corn and corn improvement). Inbred lines are classified into heterotic groups, and are further subdivided into families within a heterotic group, based on several criteria such as pedigree, molecular marker-based associations, and performance in hybrid combinations (Smith et al. (1990) Theor. Appl. Gen. 80:833-840). The two most widely used heterotic groups in the United States are referred to as “Iowa Stiff Stalk Synthetic” (also referred to herein as “stiff stalk”) and “Lancaster” or “Lancaster Sure Crop” (sometimes referred to as NSS, or non-Stiff Stalk).

Some heterotic groups possess the traits needed to be a female parent, and others, traits for a male parent. For example, in maize, yield results from public inbreds released from a population called BSSS (Iowa Stiff Stalk Synthetic population) has resulted in these inbreds and their derivatives becoming the female pool in the central Corn Belt. BSSS inbreds have been crossed with other inbreds, e.g. SD 105 and Maiz Amargo, and this general group of materials has become known as Stiff Stalk Synthetics (SSS) even though not all of the inbreds are derived from the original BSSS population (Mikel and Dudley (2006) Crop Sci: 46:1193-1205). By default, all other inbreds that combine well with the SSS inbreds have been assigned to the male pool, which for lack of a better name has been designated as NSS, i.e. Non-Stiff Stalk. This group includes several major heterotic groups such as Lancaster Surecrop, Iodent, and Leaming Corn.

The term “homogeneity” indicates that members of a group have the same genotype at one or more specific loci.

The term “hybrid” refers to the progeny obtained between the crossing of at least two genetically dissimilar parents.

The term “inbred” refers to a line that has been bred for genetic homogeneity.

The term “indel” refers to an insertion or deletion, wherein one line may be referred to as having an inserted nucleotide or piece of DNA relative to a second line, or the second line may be referred to as having a deleted nucleotide or piece of DNA relative to the first line.

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. Offspring comprising the desired allele may be repeatedly backcrossed to a line having a desired genetic background and selected for the desired allele, to result in the allele becoming fixed in a selected genetic background.

The process of “introgressing” is often referred to as “backcrossing” when the process is repeated two or more times.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor.

As used herein, the term “linkage” is used to describe the degree with which one marker locus is associated with another marker locus or some other locus. The linkage relationship between a molecular marker and a locus affecting a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units (or cM) of a single meiosis map (a genetic map based on a population that has undergone one round of meiosis, such as e.g. an F2; the IBM2 maps consist of multiple meiosis). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “in proximity to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 510% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

Linkage disequilibrium is most commonly assessed using the measure r², which is calculated using the formula described by Hill, W. G. and Robertson, A, Theor. Appl. Genet. 38:226-231(1968). When r²=1, complete LD exists between the two marker loci, meaning that the markers have not been separated by recombination and have the same allele frequency. The r² value will be dependent on the population used. Values for r² above ⅓ indicate sufficiently strong LD to be useful for mapping (Ardlie et al., Nature Reviews Genetics 3:299-309 (2002)). Hence, alleles are in linkage disequilibrium when r² values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

A “locus” is a position on a chromosome, e.g. where a nucleotide, gene, sequence, or marker is located.

The “logarithm of odds (LOD) value” or “LOD score” (Risch, Science 255:803-804 (1992)) is used in genetic interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage. LOD scores can also be used to show the strength of association between marker loci and quantitative traits in “quantitative trait loci” mapping. In this case, the LOD score's size is dependent on the closeness of the marker locus to the locus affecting the quantitative trait, as well as the size of the quantitative trait effect.

The term “plant” includes whole plants, plant cells, plant protoplast, plant cell or tissue culture from which plants can be regenerated, plant calli, plant clumps and plant cells that are intact in plants or parts of plants, such as seeds, flowers, cotyledons, leaves, stems, buds, roots, root tips and the like. As used herein, a “modified plant” means any plant that has a genetic change due to human intervention. A modified plant may have genetic changes introduced through plant transformation, genome editing, or conventional plant breeding

A “marker” is a means of finding a position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. A DNA marker, or a genetic marker, may also be used to describe the gene, DNA sequence or nucleotide on the chromosome itself (rather than the components used to detect the gene or DNA sequence) and is often used when that DNA marker is associated with a particular trait in human genetics (e.g. a marker for breast cancer). The term marker locus is the locus (gene, sequence or nucleotide) that the marker detects.

Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population.

“Marker assisted selection” (of MAS) is a process by which individual plants are selected based on marker genotypes.

“Marker assisted counter-selection” is a process by which marker genotypes are used to identify plants that will not be selected, allowing them to be removed from a breeding program or planting.

A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

An allele “negatively” correlates with a trait when it is linked to it and when presence of the allele is an indicator that a desired trait or trait form will not occur in a plant comprising the allele.

The term “phenotype”, “phenotypic trait”, or “trait” can refer to the observable expression of a gene or series of genes. The phenotype can be observable to the naked eye, or by any other means of evaluation, e.g., weighing, counting, measuring (length, width, angles, etc.), microscopy, biochemical analysis, or an electromechanical assay. In some cases, a phenotype is directly controlled by a single gene or genetic locus, i.e., a “single gene trait” or a “simply inherited trait”. In the absence of large levels of environmental variation, single gene traits can segregate in a population to give a “qualitative” or “discrete” distribution, i.e. the phenotype falls into discrete classes. In other cases, a phenotype is the result of several genes and can be considered a “multigenic trait” or a “complex trait”. Multigenic traits segregate in a population to give a “quantitative” or “continuous” distribution, i.e. the phenotype cannot be separated into discrete classes. Both single gene and multigenic traits can be affected by the environment in which they are being expressed, but multigenic traits tend to have a larger environmental component.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”.

A “production marker” or “production SNP marker” is a marker that has been developed for high-throughput purposes. Production SNP markers are developed to detect specific polymorphisms and are designed for use with a variety of chemistries and platforms.

The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question.

A “reference sequence” or a “consensus sequence” is a defined sequence used as a basis for sequence comparison. The reference sequence for a marker is obtained by sequencing a number of lines at the locus, aligning the nucleotide sequences in a sequence alignment program (e.g. Sequencher), and then obtaining the most common nucleotide sequence of the alignment. Polymorphisms found among the individual sequences are annotated within the consensus sequence. A reference sequence is not usually an exact copy of any individual DNA sequence, but represents an amalgam of available sequences and is useful for designing primers and probes to polymorphisms within the sequence.

An “unfavorable allele” of a marker is a marker allele that segregates with the unfavorable plant phenotype, therefore providing the benefit of identifying plants that can be removed from a breeding program or planting.

The term “yield” refers to the productivity per unit area of a particular plant product of commercial value. Yield is affected by both genetic and environmental factors. “Agronomics,” “agronomic traits,” and “agronomic performance” refer to the traits (and underlying genetic elements) of a given plant variety that contribute to yield over the course of growing season.

Individual agronomic traits include emergence vigor, vegetative vigor, stress tolerance, disease resistance or tolerance, herbicide resistance, branching, flowering, seed set, seed size, seed density, standability, threshability and the like. Yield is, therefore, the final culmination of all agronomic traits.

Marker loci that demonstrate statistically significant co-segregation with a disease resistance trait that confers broad resistance against a specified disease or diseases are provided herein. Detection of these loci or additional linked loci and the resistance gene may be used in marker assisted selection as part of a breeding program to produce plants that have resistance to a disease or diseases.

### Genetic Mapping

It has been recognized for quite some time that specific genetic loci correlating with particular phenotypes, such as disease resistance, can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection, or MAS).

A variety of methods are available for detecting molecular markers or clusters of molecular markers that co-segregate with a trait of interest, such as a disease resistance trait. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference. Two such methods used to detect trait loci of interest are: 1) population-based association analysis (i.e. association mapping) and 2) traditional linkage analysis.

### Association Mapping

Understanding the extent and patterns of linkage disequilibrium (LD) in the genome is a prerequisite for developing efficient association approaches to identify and map quantitative trait loci (QTL). Linkage disequilibrium (LD) refers to the non-random association of alleles in a collection of individuals. When LD is observed among alleles at linked loci, it is measured as LD decay across a specific region of a chromosome. The extent of the LD is a reflection of the recombinational history of that region. The average rate of LD decay in a genome can help predict the number and density of markers that are required to undertake a genome-wide association study and provides an estimate of the resolution that can be expected.

Association or LD mapping aims to identify significant genotype-phenotype associations. It has been exploited as a powerful tool for fine mapping in outcrossing species such as humans (Corder et al. (1994) “Protective effect of apolipoprotein-E type-2 allele for late-onset Alzheimer-disease,” Nat Genet 7:180-184; Hastbacka et al. (1992) “Linkage disequilibrium mapping in isolated founder populations: diastrophic dysplasia in Finland,” Nat Genet 2:204-211; Kerem et al. (1989) “Identification of the cystic fibrosis gene: genetic analysis,” Science 245:1073-1080) and maize (Remington et al., (2001) “Structure of linkage disequilibrium and phenotype associations in the maize genome,” Proc Natl Acad Sci USA 98:11479-11484; Thornsberry et al. (2001) “Dwarf8 polymorphisms associate with variation in flowering time,” Nat Genet 28:286-289; reviewed by Flint-Garcia et al. (2003) “Structure of linkage disequilibrium in plants,” Annu Rev Plant Biol. 54:357-374), where recombination among heterozygotes is frequent and results in a rapid decay of LD. In inbreeding species where recombination among homozygous genotypes is not genetically detectable, the extent of LD is greater (i.e., larger blocks of linked markers are inherited together) and this dramatically enhances the detection power of association mapping (Wall and Pritchard (2003) “Haplotype blocks and linkage disequilibrium in the human genome,” Nat Rev Genet 4:587-597).

The recombinational and mutational history of a population is a function of the mating habit as well as the effective size and age of a population. Large population sizes offer enhanced possibilities for detecting recombination, while older populations are generally associated with higher levels of polymorphism, both of which contribute to observably accelerated rates of LD decay. On the other hand, smaller effective population sizes, e.g., those that have experienced a recent genetic bottleneck, tend to show a slower rate of LD decay, resulting in more extensive haplotype conservation (Flint-Garcia et al. (2003) “Structure of linkage disequilibrium in plants,” Annu Rev Plant Biol. 54:357-374).

Elite breeding lines provide a valuable starting point for association analyses. Association analyses use quantitative phenotypic scores (e.g., disease tolerance rated from one to nine for each line) in the analysis (as opposed to looking only at tolerant versus resistant allele frequency distributions in intergroup allele distribution types of analysis). The availability of detailed phenotypic performance data collected by breeding programs over multiple years and environments for a large number of elite lines provides a valuable dataset for genetic marker association mapping analyses. This paves the way for a seamless integration between research and application and takes advantage of historically accumulated data sets. However, an understanding of the relationship between polymorphism and recombination is useful in developing appropriate strategies for efficiently extracting maximum information from these resources.

This type of association analysis neither generates nor requires any map data, but rather is independent of map position. This analysis compares the plants' phenotypic score with the genotypes at the various loci. Subsequently, any suitable map (for example, a composite map) can optionally be used to help observe distribution of the identified QTL markers and/or QTL marker clustering using previously determined map locations of the markers.

The same principles underlie traditional linkage analysis; however, LD is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

Marker loci that demonstrate statistically significant co-segregation with a disease resistance trait, as determined by traditional linkage analysis and by whole genome association analysis, are provided herein. Detection of these loci or additional linked loci can be used in marker assisted breeding programs to produce plants having disease resistance.

Activities in marker assisted breeding programs may include but are not limited to: selecting among new breeding populations to identify which population has the highest frequency of favorable nucleic acid sequences based on historical genotype and agronomic trait associations, selecting favorable nucleic acid sequences among progeny in breeding populations, selecting among parental lines based on prediction of progeny performance, and advancing lines in germplasm improvement activities based on presence of favorable nucleic acid sequences.

### Chromosomal Intervals

Chromosomal intervals that correlate with the disease resistance trait are provided. A variety of methods are available for identifying chromosomal intervals. The boundaries of such chromosomal intervals are drawn to encompass markers that will be linked to the gene(s) controlling the trait of interest. In other words, the chromosomal interval is drawn such that any marker that lies within that interval (including the terminal markers that define the boundaries of the interval) can be used as a marker for a disease resistance trait.

Conversely, e.g., if two markers in close proximity show co-segregation with the desired phenotypic trait, it is sometimes unclear if each of those markers identify the same gene or two different gene or multiple genes. Regardless, knowledge of how many genes are in a particular physical/genomic interval is not necessary to make or practice that which is presented in the current disclosure.

Chromosomal intervals can also be defined by markers that are linked to (show linkage disequilibrium with) a disease resistant gene, and r² is a common measure of linkage disequilibrium (LD) in the context of association studies. If the r² value of LD between a chromosome 7 marker locus in an interval of interest and another chromosome 7 marker locus in close proximity is greater than ⅓ (Ardlie et al., Nature Reviews Genetics 3:299-309 (2002)), the loci are in linkage disequilibrium with one another.

### Markers and Linkage Relationships

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker and a locus for Anthracnose Stalk Rot resistance disclosed herein) display a recombination frequency of about 1% or less, e.g., about 0.75% or less, more preferably about 0.5% or less, or yet more preferably about 0.25% or less. Thus, the marker is about 10 cM, 9 cM, 8 cM, 7 cM, 6 cM, 5 cM, 4 cM, 3 cM, 2 cM, 1 cM, 0.75 cM, 0.5 cM or 0.25 cM or less from the Anthracnose Stalk Rot resistance gene disclosed herein. Put another way, two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are said to be “proximal to” each other.

Although particular marker alleles can co-segregate with the disease resistance trait, it is important to note that the marker locus is not necessarily responsible for the expression of the disease resistance phenotype. For example, it is not a requirement that the marker polynucleotide sequence be part of a gene that is responsible for the disease resistant phenotype (for example, is part of the gene open reading frame). The association between a specific marker allele and the disease resistance trait is due to the original “coupling” linkage phase between the marker allele and the allele in the ancestral line from which the allele originated. Eventually, with repeated recombination, crossing over events between the marker and genetic locus can change this orientation. For this reason, the favorable marker allele may change depending on the linkage phase that exists within the parent having resistance to the disease that is used to create segregating populations. This does not change the fact that the marker can be used to monitor segregation of the phenotype. It only changes which marker allele is considered favorable in a given segregating population.

Methods presented herein include detecting the presence of one or more marker alleles associated with disease resistance in a plant and then identifying and/or selecting plants that have favorable alleles at those marker loci. Markers have been identified herein as being associated with the disease resistance trait and hence can be used to predict disease resistance in a plant. Any marker within 50 cM, 40 cM, 30 cM, 20 cM, 15 cM, 10 cM, 9 cM, 8 cM, 7 cM, 6 cM, 5 cM, 4 cM, 3 cM, 2 cM, 1 cM, 0.75 cM, 0.5 cM or 0.25 cM (based on a single meiosis based genetic map) could also be used to predict disease resistance in a plant.

### Marker Assisted Selection

Molecular markers can be used in a variety of plant breeding applications (e.g. see Staub et al. (1996) Hortscience 31: 729-741; Tanksley (1983) Plant Molecular Biology Reporter. 1: 3-8). One of the main areas of interest is to increase the efficiency of backcrossing and introgressing genes using marker-assisted selection (MAS). A molecular marker that demonstrates linkage with a locus affecting a desired phenotypic trait provides a useful tool for the selection of the trait in a plant population. This is particularly true where the phenotype is hard to assay. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. In some embodiments, the methods disclosed herein produce a marker in a disease resistance gene, wherein the gene was identified by inferring genomic location from clustering of conserved domains or a clustering analysis.

When a gene is introgressed by MAS, it is not only the gene that is introduced but also the flanking regions (Gepts. (2002). Crop Sci; 42: 1780-1790). This is referred to as “linkage drag.” In the case where the donor plant is highly unrelated to the recipient plant, these flanking regions carry additional genes that may code for agronomically undesirable traits. Linkage drag may also result in reduced yield or other negative agronomic characteristics even after multiple cycles of backcrossing into the elite line. This is also sometimes referred to as “yield drag.” The size of the flanking region can be decreased by additional backcrossing, although this is not always successful, as breeders do not have control over the size of the region or the recombination breakpoints (Young et al. (1998) Genetics 120:579-585). In classical breeding it is usually only by chance that recombinations are selected that contribute to a reduction in the size of the donor segment (Tanksley et al. (1989). Biotechnology 7: 257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will allow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The key components to the implementation of MAS are: (i) Defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure, as well as other marker types such as SSRs and FLPs, can be used in marker assisted selection protocols.

SSRs can be defined as relatively short runs of tandemly repeated DNA with lengths of 6 bp or less (Tautz (1989) Nucleic Acid Research 17: 6463-6471; Wang et al. (1994) Theoretical and Applied Genetics, 88:1-6). Polymorphisms arise due to variation in the number of repeat units, probably caused by slippage during DNA replication (Levinson and Gutman (1987)Mol Biol Evol 4: 203-221). The variation in repeat length may be detected by designing PCR primers to the conserved non-repetitive flanking regions (Weber and May (1989) Am J Hum Genet. 44:388-396). SSRs are highly suited to mapping and MAS as they are multi-allelic, codominant, reproducible and amenable to high throughput automation (Rafalski et al. (1996) Generating and using DNA markers in plants. In: Non-mammalian genomic analysis: a practical guide. Academic press. pp 75-135).

Various types of SSR markers can be generated, and SSR profiles can be obtained by gel electrophoresis of the amplification products. Scoring of marker genotype is based on the size of the amplified fragment.

Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al. (2002). Plant Mol Biol 48, 539-547; Rafalski (2002b), supra).

SNP markers detect single base pair nucleotide substitutions. Of all the molecular marker types, SNPs are the most abundant, thus having the potential to provide the highest genetic map resolution (Bhattramakki et al. 2002 Plant Molecular Biology 48:539-547). SNPs can be assayed at an even higher level of throughput than SSRs, in a so-called ‘ultra-high-throughput’ fashion, as SNPs do not require large amounts of DNA and automation of the assay may be straight-forward. SNPs also have the promise of being relatively low-cost systems. These three factors together make SNPs highly attractive for use in MAS. Several methods are available for SNP genotyping, including but not limited to, hybridization, primer extension, oligonucleotide ligation, nuclease cleavage, minisequencing, and coded spheres. Such methods have been reviewed in: Gut (2001) Hum Mutat 17 pp. 475-492; Shi (2001) Clin Chem 47, pp. 164-172; Kwok (2000) Pharmacogenomics 1, pp. 95-100; and Bhattramakki and Rafalski (2001) Discovery and application of single nucleotide polymorphism markers in plants. In: R. J. Henry, Ed, Plant Genotyping: The DNA Fingerprinting of Plants, CABI Publishing, Wallingford. A wide range of commercially available technologies utilize these and other methods to interrogate SNPs including Masscode™ (Qiagen), INVADER®. (Third Wave Technologies) and Invader PLUS®, SNAPSHOT®. (Applied Biosystems), TAQMAN®. (Applied Biosystems) and BEADARRAYS®. (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than single SNPs and can be more descriptive of any particular genotype. For example, a single SNP may be allele “T” for a specific line or variety with disease resistance, but the allele ‘T’ might also occur in the breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. Using automated high throughput marker detection platforms makes this process highly efficient and effective.

Many of the markers presented herein can readily be used as single nucleotide polymorphic (SNP) markers to select for the NLR 04. Using PCR, the primers are used to amplify DNA segments from individuals (preferably inbred) that represent the diversity in the population of interest. The PCR products are sequenced directly in one or both directions. The resulting sequences are aligned and polymorphisms are identified. The polymorphisms are not limited to single nucleotide polymorphisms (SNPs), but also include indels, CAPS, SSRs, and VNTRs (variable number of tandem repeats). Specifically, with respect to the fine map information described herein, one can readily use the information provided herein to obtain additional polymorphic SNPs (and other markers) within the region amplified by the primers disclosed herein. Markers within the described map region can be hybridized to BACs or other genomic libraries, or electronically aligned with genome sequences, to find new sequences in the same approximate location as the described markers.

In addition to SSR's, FLPs and SNPs, as described above, other types of molecular markers are also widely used, including but not limited to expressed sequence tags (ESTs), SSR markers derived from EST sequences, randomly amplified polymorphic DNA (RAPD), and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the species, or even across other species that have been genetically or physically aligned.

In general, MAS uses polymorphic markers that have been identified as having a significant likelihood of co-segregation with a trait such as the ANTROT disease resistance trait. Such markers are presumed to map near a gene or genes that give the plant its disease resistant phenotype, and are considered indicators for the desired trait, or markers. Plants are tested for the presence of a desired allele in the marker, and plants containing a desired genotype at one or more loci are expected to transfer the desired genotype, along with a desired phenotype, to their progeny. Thus, plants with ANTROT disease resistance may be selected for by detecting one or more marker alleles, and in addition, progeny plants derived from those plants can also be selected. Hence, a plant containing a desired genotype in a given chromosomal region (i.e. a genotype associated with disease resistance) is obtained and then crossed to another plant. The progeny of such a cross would then be evaluated genotypically using one or more markers and the progeny plants with the same genotype in a given chromosomal region would then be selected as having disease resistance.

The skilled artisan would expect that there might be additional polymorphic sites at marker loci in and around a chromosome marker identified by the methods disclosed herein, wherein one or more polymorphic sites is in linkage disequilibrium (LD) with an allele at one or more of the polymorphic sites in the haplotype and thus could be used in a marker assisted selection program to introgress a gene allele or genomic fragment of interest. Two particular alleles at different polymorphic sites are said to be in LD if the presence of the allele at one of the sites tends to predict the presence of the allele at the other site on the same chromosome (Stevens, Mol. Diag. 4:309-17 (1999)). The marker loci can be located within 5 cM, 2 cM, or 1 cM (on a single meiosis based genetic map) of the disease resistance trait QTL.

The skilled artisan would understand that allelic frequency (and hence, haplotype frequency) can differ from one germplasm pool to another. Germplasm pools vary due to maturity differences, heterotic groupings, geographical distribution, etc. As a result, SNPs and other polymorphisms may not be informative in some germplasm pools.

### Plant Compositions

Plants identified, modified, and/or selected by any of the methods described above are also of interest.

### Proteins and Variants and Fragments Thereof

NLR 04 polypeptides are encompassed by the disclosure. “NLR 04 polypeptide” and “NLR 04 protein” as used herein interchangeably refers to a polypeptide(s) having ANTROT resistance activity, and is sufficiently identical to the NLR 04 polypeptide of SEQ ID NO: 30. A variety of NLR 04 polypeptides are contemplated.

“Sufficiently identical” is used herein to refer to an amino acid sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity. In some embodiments the sequence identity is against the full-length sequence of a polypeptide. The term “about” when used herein in context with percent sequence identity means +/−1.0%.

A “recombinant protein” is used herein to refer to a protein that is no longer in its natural environment, for example in vitro or in a recombinant bacterial or plant host cell; a protein that is expressed from a polynucleotide that has been edited from its native version; or a protein that is expressed from a polynucleotide in a different genomic position relative to the native sequence.

“Substantially free of cellular material” as used herein refers to a polypeptide including preparations of protein having less than about 30%, 20%, 10% or 5% (by dry weight) of non-target protein (also referred to herein as a “contaminating protein”).

“Fragments” or “biologically active portions” include polypeptide or polynucleotide fragments comprising sequences sufficiently identical to an NLR 04 polypeptide or polynucleotide, respectively, and that exhibit disease resistance when expressed in a plant.

“Variants” as used herein refers to proteins or polypeptides having an amino acid sequence that is at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or greater identical to the parental amino acid sequence.

In some embodiments a NLR 04 polypeptide comprises an amino acid sequence having at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity to the full length or a fragment of the amino acid sequence of SEQ ID NO: 30.

Methods for such manipulations are generally known in the art. For example, amino acid sequence variants of a NLR 04 polypeptide may be prepared by mutations in the DNA. This may also be accomplished by one of several forms of mutagenesis, such as for example site-specific double strand break technology, and/or in directed evolution. In some aspects, the changes encoded in the amino acid sequence will not substantially affect the function of the protein. Such variants will possess the desired activity. However, it is understood that the ability of an NLR 04 polypeptide to confer disease resistance may be improved by the use of such techniques upon the compositions of this disclosure.

### Nucleic Acid Molecules and Variants and Fragments Thereof

Isolated or recombinant nucleic acid molecules comprising nucleic acid sequences encoding NLR 04 polypeptide or biologically active portions thereof, as well as nucleic acid molecules sufficient for use as hybridization probes to identify nucleic acid molecules encoding proteins with regions of sequence homology are provided. As used herein, the term “nucleic acid molecule” refers to DNA molecules (e.g., recombinant DNA, cDNA, genomic DNA, plastid DNA, mitochondrial DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule (or DNA) is used herein to refer to a nucleic acid sequence (or DNA) that is no longer in its natural environment, for example in vitro. A “recombinant” nucleic acid molecule (or DNA) is used herein to refer to a nucleic acid sequence (or DNA) that is in a recombinant bacterial or plant host cell; has been edited from its native sequence; or is located in a different location than the native sequence. In some embodiments, an “isolated” or “recombinant” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the disclosure, “isolated” or “recombinant” when used to refer to nucleic acid molecules excludes isolated chromosomes. For example, in various embodiments, the recombinant nucleic acid molecules encoding NLR 04 polypeptides can contain less than about 5 kb, 4 kb, 3 kb, 2 kb, 1 kb, 0.5 kb or 0.1 kb of nucleic acid sequences that naturally flank the nucleic acid molecule in genomic DNA of the cell from which the nucleic acid is derived.

In some embodiments an isolated nucleic acid molecule encoding NLR 04 polypeptide has one or more change in the nucleic acid sequence compared to the native or genomic nucleic acid sequence. In some embodiments the change in the native or genomic nucleic acid sequence includes but is not limited to: changes in the nucleic acid sequence due to the degeneracy of the genetic code; changes in the nucleic acid sequence due to the amino acid substitution, insertion, deletion and/or addition compared to the native or genomic sequence; removal of one or more intron; deletion of one or more upstream or downstream regulatory regions; and deletion of the 5′ and/or 3′ untranslated region associated with the genomic nucleic acid sequence. In some embodiments the nucleic acid molecule encoding an NLR 04 polypeptide is a non-genomic sequence.

A variety of polynucleotides that encode NLR 04 polypeptides or related proteins are contemplated. Such polynucleotides are useful for production of NLR 04 polypeptides in host cells when operably linked to a suitable promoter, transcription termination and/or polyadenylation sequences. Such polynucleotides are also useful as probes for isolating homologous or substantially homologous polynucleotides that encode NLR 04 polypeptides or related proteins.

In some embodiments the nucleic acid molecule encoding an NLR 04 polypeptide is a polynucleotide having the sequence set forth in, and variants, fragments and complements thereof. “Complement” is used herein to refer to a nucleic acid sequence that is sufficiently complementary to a given nucleic acid sequence such that it can hybridize to the given nucleic acid sequence to thereby form a stable duplex. “Polynucleotide sequence variants” is used herein to refer to a nucleic acid sequence that except for the degeneracy of the genetic code encodes the same polypeptide.

In some embodiments the nucleic acid molecule encoding the NLR 04 polypeptide is a non-genomic nucleic acid sequence. As used herein a “non-genomic nucleic acid sequence” or “non-genomic nucleic acid molecule” or “non-genomic polynucleotide” refers to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; optimization of the nucleic acid sequence for expression in plants; changes in the nucleic acid sequence to introduce at least one amino acid substitution, insertion, deletion and/or addition compared to the native or genomic sequence; removal of one or more intron associated with the genomic nucleic acid sequence; insertion of one or more heterologous introns; deletion of one or more upstream or downstream regulatory regions associated with the genomic nucleic acid sequence; insertion of one or more heterologous upstream or downstream regulatory regions; deletion of the 5′ and/or 3′ untranslated region associated with the genomic nucleic acid sequence; insertion of a heterologous 5′ and/or 3′ untranslated region; and modification of a polyadenylation site. In some embodiments the non-genomic nucleic acid molecule is a synthetic nucleic acid sequence.

In some embodiments the nucleic acid molecule encoding an NLR 04 polypeptide disclosed herein is a non-genomic polynucleotide having a nucleotide sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity, to the nucleic acid sequence of SEQ ID NO: 30.

Nucleic acid molecules that are fragments of these nucleic acid sequences encoding NLR 04 polypeptides are also encompassed by the embodiments. “Fragment” as used herein refers to a portion of the nucleic acid sequence encoding an NLR 04 polypeptide. A fragment of a nucleic acid sequence may encode a biologically active portion of an NLR 04 polypeptide or it may be a fragment that can be used as a hybridization probe or PCR primer using methods disclosed below. Nucleic acid molecules that are fragments of a nucleic acid sequence encoding an NLR 04 polypeptide comprise at least about 150, 180, 210, 240, 270, 300, 330, 360, 400, 450, or 500 contiguous nucleotides or up to the number of nucleotides present in a full-length nucleic acid sequence encoding a NLR 04 polypeptide identified by the methods disclosed herein, depending upon the intended use. “Contiguous nucleotides” is used herein to refer to nucleotide residues that are immediately adjacent to one another. Fragments of the nucleic acid sequences of the embodiments will encode protein fragments that retain the biological activity of the NLR 04 polypeptide and, hence, retain disease resistance. “Retains disease resistance” is used herein to refer to a polypeptide having at least about 10%, at least about 30%, at least about 50%, at least about 70%, 80%, 90%, 95% or higher of the disease resistance of the full-length NLR 04 polypeptide as set forth in SEQ ID NO: 30.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

In some embodiments a NLR 04 polynucleotide encodes a NLR 04 polypeptide comprising an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 30. In some embodiments, a NLR 04 polynucleotide comprises genomic sequence, including introns, regulatory elements, and untranslated regions.

The embodiments also encompass nucleic acid molecules encoding NLR 04 polypeptide variants. “Variants” of NLR 04 polypeptide encoding nucleic acid sequences include those sequences that encode the NLR 04 polypeptides identified by the methods disclosed herein, but that differ conservatively because of the degeneracy of the genetic code as well as those that are sufficiently identical as discussed above. Naturally occurring allelic variants can be identified with the use of well-known molecular biology techniques, such as polymerase chain reaction (PCR) and hybridization techniques as outlined below. Variant nucleic acid sequences also include synthetically derived nucleic acid sequences that have been generated, for example, by using site-directed mutagenesis but which still encode the NLR 04 polypeptides disclosed herein.

The skilled artisan will further appreciate that changes can be introduced by mutation of the nucleic acid sequences thereby leading to changes in the amino acid sequence of the encoded NLR 04 polypeptides, without altering the biological activity of the proteins. Thus, variant nucleic acid molecules can be created by introducing one or more nucleotide substitutions, additions and/or deletions into the corresponding nucleic acid sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleic acid sequences are also encompassed by the present disclosure.

Alternatively, variant nucleic acid sequences can be made by introducing mutations randomly along all or part of the coding sequence, such as by saturation mutagenesis, and the resultant mutants can be screened for ability to confer activity to identify mutants that retain activity. Following mutagenesis, the encoded protein can be expressed recombinantly, and the activity of the protein can be determined using standard assay techniques.

The polynucleotides of the disclosure and fragments thereof are optionally used as substrates for a variety of recombination and recursive recombination reactions, in addition to standard cloning methods as set forth in, e.g., Ausubel, Berger and Sambrook, i.e., to produce additional polypeptide homologues and fragments thereof with desired properties. A variety of such reactions are known. Methods for producing a variant of any nucleic acid listed herein comprising recursively recombining such polynucleotide with a second (or more) polynucleotide, thus forming a library of variant polynucleotides are also embodiments of the disclosure, as are the libraries produced, the cells comprising the libraries and any recombinant polynucleotide produced by such methods. Additionally, such methods optionally comprise selecting a variant polynucleotide from such libraries based on activity, as is wherein such recursive recombination is done in vitro or in vivo.

A variety of diversity generating protocols, including nucleic acid recursive recombination protocols are available. The procedures can be used separately, and/or in combination to produce one or more variants of a nucleic acid or set of nucleic acids, as well as variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids with or which confer desirable properties or that encode proteins with or which confer desirable properties. Following diversification by one or more of the methods herein or otherwise available to one of skill, any nucleic acids that are produced can be selected for a desired activity or property, e.g. such activity at a desired pH, etc. This can include identifying any activity that can be detected, for example, in an automated or automatable format, by any of the assays in the art. A variety of related (or even unrelated) properties can be evaluated, in serial or in parallel, at the discretion of the practitioner.

The nucleotide sequences of the embodiments can also be used to isolate corresponding sequences from a different source. In this manner, methods such as PCR, hybridization, and the like can be used to identify such sequences based on their sequence homology to the sequences identified by the methods disclosed herein. Sequences that are selected based on their sequence identity to the entire sequences set forth herein or to fragments thereof are encompassed by the embodiments. Such sequences include sequences that are orthologs of the sequences. The term “orthologs” refers to genes derived from a common ancestral gene and which are found in different species as a result of speciation. Genes found in different species are considered orthologs when their nucleotide sequences and/or their encoded protein sequences share substantial identity as defined elsewhere herein.

In a PCR approach, oligonucleotide primers can be designed for use in PCR reactions to amplify corresponding DNA sequences from cDNA or genomic DNA extracted from any organism of interest. Methods for designing PCR primers and PCR cloning are disclosed in Sambrook, et al., (1989) Molecular Cloning: A Laboratory Manual (2d ed., Cold Spring Harbor Laboratory Press, Plainview, New York), hereinafter “Sambrook”. See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization methods, all or part of the nucleic acid sequence can be used to screen cDNA or genomic libraries. Methods for construction of such cDNA and genomic libraries are disclosed in Sambrook and Russell, (2001), supra. The so-called hybridization probes may be genomic DNA fragments, cDNA fragments, RNA fragments or other oligonucleotides and may be labeled with a detectable group such as 32P or any other detectable marker, such as other radioisotopes, a fluorescent compound, an enzyme or an enzyme co-factor. Probes for hybridization can be made by labeling synthetic oligonucleotides based on the known polypeptide-encoding nucleic acid sequences disclosed herein. Degenerate primers designed on the basis of conserved nucleotides or amino acid residues in the nucleic acid sequence or encoded amino acid sequence can additionally be used. The probe typically comprises a region of nucleic acid sequence that hybridizes under stringent conditions to at least about 12, at least about 25, at least about 50, 75, 100, 125, 150, 175 or 200 consecutive nucleotides of nucleic acid sequences encoding polypeptides or a fragment or variant thereof. Methods for the preparation of probes for hybridization and stringency conditions are disclosed in Sambrook and Russell, (2001), supra.

### Nucleotide Constructs, Expression Cassettes and Vectors

The use of the term “nucleotide constructs” herein is not intended to limit the embodiments to nucleotide constructs comprising DNA. Those of ordinary skill in the art will recognize that nucleotide constructs, particularly polynucleotides and oligonucleotides composed of ribonucleotides and combinations of ribonucleotides and deoxyribonucleotides, may also be employed in the methods disclosed herein. The nucleotide constructs, nucleic acids, and nucleotide sequences of the embodiments additionally encompass all complementary forms of such constructs, molecules, and sequences. Further, the nucleotide constructs, nucleotide molecules, and nucleotide sequences of the embodiments encompass all nucleotide constructs, molecules, and sequences which can be employed in the methods of the embodiments for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs, nucleic acids, and nucleotide sequences of the embodiments also encompass all forms of nucleotide constructs including, but not limited to, single-stranded forms, double-stranded forms, hairpins, stem-and-loop structures and the like.

A further embodiment relates to a transformed organism such as an organism selected from plant cells, bacteria, yeast, baculovirus, protozoa, nematodes and algae. The transformed organism comprises a DNA molecule of the embodiments, an expression cassette comprising the DNA molecule or a vector comprising the expression cassette, which may be stably incorporated into the genome of the transformed organism.

The sequences of the embodiments are provided in DNA constructs for expression in the organism of interest. The construct will include 5′ and 3′ regulatory sequences operably linked to a sequence of the embodiments. The term “operably linked” as used herein refers to a functional linkage between a promoter and/or a regulatory sequence and a second sequence, wherein the promoter and/or regulatory sequence initiates, mediates, and/or affects transcription of the DNA sequence corresponding to the second sequence. Generally, operably linked means that the nucleic acid sequences being linked are contiguous and where necessary to join two protein coding regions in the same reading frame. The construct may additionally contain at least one additional gene to be cotransformed into the organism. Alternatively, the additional gene(s) can be provided on multiple DNA constructs.

Such a DNA construct is provided with a plurality of restriction sites for insertion of the polypeptide gene sequence of the disclosure to be under the transcriptional regulation of the regulatory regions. The DNA construct may additionally contain selectable marker genes.

The DNA construct will generally include in the 5′ to 3′ direction of transcription: a transcriptional and translational initiation region (i.e., a promoter), a DNA sequence of the embodiments, and a transcriptional and translational termination region (i.e., termination region) functional in the organism serving as a host. The transcriptional initiation region (i.e., the promoter) may be native, analogous, foreign or heterologous to the host organism and/or to the sequence of the embodiments. Additionally, the promoter or regulatory sequence may be the natural sequence or alternatively a synthetic sequence. The term “foreign” as used herein indicates that the promoter is not found in the native organism into which the promoter is introduced. As used herein, the term “heterologous” in reference to a sequence means a sequence that originates from a foreign species or, if from the same species, is substantially modified from its native form in composition and/or genomic locus by deliberate human intervention. As used herein, a chimeric gene comprises a coding sequence operably linked to a transcription initiation region that is heterologous to the coding sequence. Where the promoter is a native or natural sequence, the expression of the operably linked sequence is altered from the wild-type expression, which results in an alteration in phenotype.

In some embodiments the DNA construct comprises a polynucleotide encoding an NLR 04 polypeptide of the embodiments. In some embodiments the DNA construct comprises a polynucleotide encoding a fusion protein comprising an NLR 04 polypeptide of the embodiments.

In some embodiments the DNA construct may also include a transcriptional enhancer sequence. As used herein, the term an “enhancer” refers to a DNA sequence which can stimulate promoter activity, and may be an innate element of the promoter or a heterologous element inserted to enhance the level or tissue-specificity of a promoter. Various enhancers include, for example, introns with gene expression enhancing properties in plants (US Patent Application Publication Number 2009/0144863, the ubiquitin intron (i.e., the maize ubiquitin intron 1 (see, for example, NCBI sequence S94464)), the omega enhancer or the omega prime enhancer (Gallie, et al., (1989) Molecular Biology of RNA ed. Cech (Liss, New York) 237-256 and Gallie, et al., (1987) Gene 60:217-25), the CaMV 35S enhancer (see, e.g., Benfey, et al., (1990) EMBO J. 9:1685-96) and the enhancers of U.S. Pat. No. 7,803,992 may also be used. The above list of transcriptional enhancers is not meant to be limiting. Any appropriate transcriptional enhancer can be used in the embodiments.

The termination region may be native with the transcriptional initiation region, may be native with the operably linked DNA sequence of interest, may be native with the plant host or may be derived from another source (i.e., foreign or heterologous to the promoter, the sequence of interest, the plant host or any combination thereof).

Convenient termination regions are available from the Ti-plasmid of A. tumefaciens, such as the octopine synthase and nopaline synthase termination regions. See also, Guerineau, et al., (1991)Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acid Res. 15:9627-9639.

Where appropriate, a nucleic acid may be optimized for increased expression in the host organism. Thus, where the host organism is a plant, the synthetic nucleic acids can be synthesized using plant-preferred codons for improved expression. See, for example, Campbell and Gown, (1990) Plant Physiol. 92:1-11 for a discussion of host-preferred usage. For example, although nucleic acid sequences of the embodiments may be expressed in both monocotyledonous and dicotyledonous plant species, sequences can be modified to account for the specific preferences and GC content preferences of monocotyledons or dicotyledons as these preferences have been shown to differ (Murray et al. (1989) Nucleic Acids Res. 17:477-498). Thus, the plant-preferred for a particular amino acid may be derived from known gene sequences from plants.

Additional sequence modifications are known to enhance gene expression in a cellular host. These include elimination of sequences encoding spurious polyadenylation signals, exon-intron splice site signals, transposon-like repeats, and other well-characterized sequences that may be deleterious to gene expression. The GC content of the sequence may be adjusted to levels average for a given cellular host, as calculated by reference to known genes expressed in the host cell. The term “host cell” as used herein refers to a cell which contains a vector and supports the replication and/or expression of the expression vector is intended. Host cells may be prokaryotic cells such as E. coli or eukaryotic cells such as yeast, insect, amphibian or mammalian cells or monocotyledonous or dicotyledonous plant cells. An example of a monocotyledonous host cell is a maize host cell. When possible, the sequence is modified to avoid predicted hairpin secondary mRNA structures.

In preparing the expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, e.g., transitions and transversions, may be involved.

A number of promoters can be used in the practice of the embodiments. The promoters can be selected based on the desired outcome. The nucleic acids can be combined with constitutive, tissue-preferred, inducible or other promoters for expression in the host organism.

### Plant Transformation

The methods of the embodiments involve introducing a polypeptide or polynucleotide into a plant. “Introducing” is as used herein means presenting to the plant the polynucleotide or polypeptide in such a manner that the sequence gains access to the interior of a cell of the plant. The methods of the embodiments do not depend on a particular method for introducing a polynucleotide or polypeptide into a plant, only that the polynucleotide(s) or polypeptide(s) gains access to the interior of at least one cell of the plant. Methods for introducing polynucleotide(s) or polypeptide(s) into plants include, but are not limited to, stable transformation methods, transient transformation methods, and virus-mediated methods.

“Stable transformation” as used herein means that the nucleotide construct introduced into a plant integrates into the genome of the plant and is capable of being inherited by the progeny thereof “Transient transformation” as used herein means that a polynucleotide is introduced into the plant and does not integrate into the genome of the plant or a polypeptide is introduced into a plant. “Plant” as used herein refers to whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells and pollen).

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (U.S. Pat. Nos. 5,563,055 and 5,981,840), direct gene transfer (Paszkowski, et al., (1984) EMBO J 3:2717-2722) and ballistic particle acceleration (see, for example, U.S. Pat. Nos. 4,945,050; 5,879,918; 5,886,244 and 5,932,782; Tomes, et al., (1995) in Plant Cell, Tissue, and Organ Culture: Fundamental Methods, ed. Gamborg and Phillips, (Springer-Verlag, Berlin) and McCabe, et al., (1988) Biotechnology 6:923-926) and Lecl transformation (WO 00/28058). For potato transformation see, Tu, et al., (1998) Plant Molecular Biology 37:829-838 and Chong, et al., (2000) Transgenic Research 9:71-78. Additional transformation procedures can be found in Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens).

Methods to Introduce Genome Editing Technologies into Plants

In some embodiments, polynucleotide compositions can be introduced into the genome of a plant using genome editing technologies, or previously introduced polynucleotides in the genome of a plant may be edited using genome editing technologies. For example, the identified polynucleotides can be introduced into a desired location in the genome of a plant through the use of double-stranded break technologies such as TALENs, meganucleases, zinc finger nucleases, CRISPR-Cas, and the like. For example, the identified polynucleotides can be introduced into a desired location in a genome using a CRISPR-Cas system, for the purpose of site-specific insertion. The desired location in a plant genome can be any desired target site for insertion, such as a genomic region amenable for breeding or may be a target site located in a genomic window with an existing trait of interest. Existing traits of interest could be either an endogenous trait or a previously introduced trait.

In some embodiments, NLR 04 has been identified in a genome, genome editing technologies may be used to alter or modify the polynucleotide sequence. Site specific modifications that can be introduced into the NLR 04 allele polynucleotide include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. Such technologies can be used to modify the previously introduced polynucleotide through the insertion, deletion or substitution of nucleotides within the introduced polynucleotide. Alternatively, double-stranded break technologies can be used to add additional nucleotide sequences to the introduced polynucleotide. Additional sequences that may be added include, additional expression elements, such as enhancer and promoter sequences. In another embodiment, genome editing technologies may be used to position additional disease resistant proteins in close proximity to the NLR 04 compositions within the genome of a plant, in order to generate molecular stacks disease resistant proteins.

An “altered target site,” “altered target sequence.” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

### EXAMPLES

The following examples are offered to illustrate, but not to limit, the claimed subject matter. It is understood that the examples and embodiments described herein are for illustrative purposes only, and persons skilled in the art will recognize various reagents or parameters that can be altered without departing from the spirit of the disclosure or the scope of the appended claims.

### Comparative Example

Creation of Population with Increased Resistance to Anthracnose Stalk Rot

Anthracnose Stalk Rot (ANTROT) caused by the fungal pathogen Colletotrichum graminicola is a destructive stalk disease of maize, responsible for consistent and significant yield losses. (Mueller et al., Plant Health Progress 2020, 238-247). As described in U.S. Patent Publication No. US2016-0355840A1, F1-derived DH mapping population between the inbreds INBRED A and INBRED B was used to identify QTL associated with resistance to anthracnose stalk rot. The inbred INBRED A is resistant to ANTROT in contrast to the inbred INBRED B. The per se score of the parents used to create this F1DH population were an ANTSUM score of 1.5 and 9.9 for INBRED A and INBRED B, respectively. The mapping population exhibited varying degrees of resistance. The populations were genotyped on a 768 SNP array specific for the NSS heterotic group. The phenotypes ANTINODES, ANTGR75, ANTSUM and ANTROT were collected in field experiments for these populations. The phenotype ANTINODES represents the number of internodes that are showing discoloration indicating infection by the pathogen and includes the internode that was inoculated. Scores range from 1 to 5 with a 1 corresponding to resistance and a 5 corresponding to susceptibility. The phenotype ANTGR75 represent the number of internodes that are showing greater than 75% discoloration. Scores range from 1 to 5 with a 1 corresponding to resistance and a 5 corresponding to susceptibility. The phenotype ANTSUM is the sum of the ANTINODES and ANTGR75 phenotypes with a range of scores are from 1 (Resistant) to 10 (Susceptible). ANTSUM is the score reported in the data tables below. The phenotype ANTROT is a score based on a count or evaluation of stalk quality with a 1 (susceptible) to 9 (resistant) score.

Associations between genotypes and phenotypes were incorporated into a QTL mapping program (R-QTL), identifying a QTL for resistance to anthracnose stalk rot on Chromosome 10 between 10-90 cM.

### Determination of Effect of South American QTL for Resistance to Anthracnose Stalk Rot in North America

Progeny from the F1DH population were sent to a North American breeding station to determine the efficacy of resistance in the INBRED A inbred with different races of the fungus Colletotrichum graminicola present in North America. The progeny with resistance scored 4.2 points better (Resistant 2.3 vs susceptible 6.5) using the ANTSUM trait.

The effect was measured again in the same F1DH population crossed with a tester, INBRED E, to determine the effect of the resistance in a hybrid setting.

The 1.5 point difference was in line with a continuation of population and marker development to narrow down the resistance QTL region. Based on genotypes (SNP calls from the F1DH material) and phenotypes collected, Table 1 provides details of the effect in this population using phenotypes from the hybrid testing. The phenotypes as a response to infection with Colletotrichum graminicola and the genotypes were analyzed with TIBCO Spotfire (v 10.10.3.3), which employs a Kruskal-Wallis methodology for comparing numeric and categorical variables to determine the p-value of the association between phenotype and genotype.

TABLE 1
Maize markers on chromosome 10 associated with anthracnose stalk rot resistance
in the hybrid phenotyping experiment (2012). The p-value presented represents the
correlation of the genotype at a given genetic position with the ANTSUM phenotype
in INBRED A. The physical positions are the public B73 genome (Version 5).

| | | Physical | | | | | SEQ | SNP |
|---|---|---|---|---|---|---|---|---|
| | | Position | RP | Donor | INBRED | INBRED | ID | Pos in |
| Marker | p-value | (B73 v5) | Allele | Allele | A | B | No. | Seq |
| PHM12565-12 | 4.0E−02 | 4,215,671 | 6.11 | 4.48 | G | A | 1 | 121 |
| PHM1822-25 | 1.5E−01 | 5,954,283 | 5.96 | 4.89 | A | C | 2 | 121 |
| PHM2828-83 | 6.8E−02 | 6,260,285 | 5.96 | 4.51 | T | C | 3 | 121 |
| PHM13727-29 | 6.8E−02 | 8,608,036 | 5.96 | 4.51 | A | C | 4 | 121 |
| PHM3922-31 | 1.2E−01 | 17,688,464 | 5.96 | 5.28 | A | G | 5 | 233 |
| PHM15854-7 | 2.2E−01 | 130,092,859 | 5.81 | 5.61 | C | T | 6 | 181 |
| PHM9369-27 | 2.3E−01 | 143,162,215 | 5.39 | 6.11 | G | A | 7 | 121 |
| PHM11874-27 | 2.2E−01 | 149,224,824 | 6.03 | 5.80 | C | T | 8 | 121 |
| PHM14796-110 | 2.2E−01 | 150,629,618 | 6.03 | 5.80 | A | G | 9 | 121 |

BC3 populations were developed in an additional susceptible background, PH1M6A (U.S. Pat. No. 8,884,128) as a recurrent parent, with the resistant locus from INBRED A selected for by marker assisted selection. The progeny from these populations were phenotyped at a NA breeding station and genotypic data was generated using Tagman markers selected for polymorphism between parents of the respective crosses and in the region of interest on Chromosome 10. Using the Kruskal-Wallis analysis, a p-value of 1.00E-030 was obtained for PZE-110006361 at position 32.9 (˜4.8 MB) on chromosome 10 (WO2016/19629A1), which is a strong association between genotype and phenotype with a subsequent refinement of the region to Chromosome 10 from ˜18-40 cM (1.7-5.6 MB) (U.S. Patent Publication No. US2016-0355840A1).

### Exome Capture Sequence Marker Development

Exome capture of INBRED A derived near isogenic line (NIL) bulks was utilized to discover SNPs attributable to genes in the QTL area whereupon there would be differences on whether or not the bulked DNA possessed the INBRED A introgression, (NIL QTL positive bulks) or if it was the recurrent parent (RP) background (NIL QTL negative bulk). Eight different bulks were made of NILs in four different RP backgrounds (PH1M1Y, INBRED C, INBRED D, and PH17JT). For each RP background there is a bulk with and a bulk without the region of interest. Reported SNPs were used to identify more markers for fine-mapping the QTL.

Sequence capture probes were used to capture DNA enriched from exonic regions of the genome, followed by Illumina short read sequencing. Reads from this dataset were used to discover additional SNPs between the donor and recurrent parents. Raw data was assembled using custom scripts which reported SNP calls against the B73 reference calls. Markers were designed for those SNPs that differed from B73 reference in all four INBRED A positive bulks, versus the INBRED A negative bulks which had the same call as the B73 reference.

Markers were designed for these SNPs and were assayed against INBRED A and the recurrent parents. Markers that were diagnostic between parents were then screened against recombinants from the BC3S2 population. These additional markers the region were used for further fine mapping.

To further refine region of interest on C10, additional markers from the 56K SNP Chip were designed as KASP markers utilizing proprietary software to design markers utilizing the SNP calls as competing forward primers with a common reverse sequence. Testing with parents of the population and subsequently testing with a small panel of recombinants yielded four markers that defined the QTL area (U.S. Patent Publication No. US2016-0355840A1).

Additional phenotyping further refined the QTL position to ˜1.7-4.4 MB (Table 2). The most significant markers were C01964-1 (p-value of 5.11 E-063) and C01957-1 (1.12 E-62). The INBRED A haplotype shows the effect of a dominant QTL as A and H alleles have similar ANTSUM scores. At C01964-1, the average ANTSUM score for individuals with the donor allele (INBRED A) was 2.6. Heterozygotes had a score of 3 and individuals with the recurrent parent (PH17JT) haplotype had a score of 6.2.

TABLE 2
Maize markers significantly associated with anthracnose stalk resistance (2014) on Chromosome
10. All significances and positions are as presented as in previous table.

| | | Physical | | | | | | SEQ | SNP |
|---|---|---|---|---|---|---|---|---|---|
| | | Position | RP | Donor | | INBRED | | ID | Pos in |
| Marker | p-value | (B73 v5) | Allele | Allele | Het | A | PH17JT | No. | Seq |
| SYN17615 | 4.8E−19 | 1,977,385 | 5.19 | 4.22 | 3.75 | C | A | 10 | 61 |
| SYN17616 | 2.45E−18 | 1,978,262 | 5.34 | 3.75 | 3.76 | G | A | 11 | 61 |
| C00429-801 | 5.59E−17 | 1,748,641 | 5.29 | 3.74 | 3.74 | T | A | 12 | 84 |
| SYN17612 | 2.88E−20 | 1,992,573 | 5.43 | 3.7 | 3.7 | T | G | 13 | 61 |
| SYN12395 | 1.13E−30 | 2,203,151 | 5.66 | 3.38 | 3.39 | A | G | 14 | 61 |
| SYN17244 | 2.35E−44 | 2,555,169 | 5.93 | 2.83 | 3.36 | T | C | 17 | 61 |
| C01964-1 | 5.11E−63 | 2,815,521 | 6.15 | 2.59 | 3.02 | T | G | 15 | 201 |
| C01957-1 | 1.12E−62 | 2,816,414 | 6.15 | 3.04 | 3.04 | C | T | 16 | 201 |
| SYN14840 | 1.49E−23 | 4,063,349 | 5.8 | 3.71 | 3.97 | G | A | 18 | 61 |
| SYN17783 | 5.27E−03 | 4,413,296 | 5.09 | 4.37 | 4.53 | A | G | 19 | 61 |
| PZE- | 2.04E−03 | 4,430,108 | 5.12 | 4.29 | 4.48 | G | T | 20 | 51 |
| 110006361 | | | | | | | | | |

Additional BC4S2 populations were generated and the resistant locus from INBRED A further selected in the PH1M6A background by marker assisted selection. A panel of Taqman SNP markers were genotyped with the additional SNP markers that proved diagnostic for these populations in the chromosome 10 region. The populations were phenotyped and associations between phenotypes and genotypes was made using the Kruskal-Wallis analysis. Table 3 shows P-values ranging between 4.96E-021 to 1.05E-25 for the markers between 2.8-3.5 MB. The strongest association was at marker PHM10 (˜3.4 MB) at 4.9E-26. The INBRED A haplotype again shows the effect of a dominant QTL as A and H alleles have similar ANTSUM scores. At PHM10, the average ANTSUM score for individuals with the donor allele (INBRED A) was 2.2. Heterozygotes had a score of 2.4 and individuals with the recurrent parent (PH1M6A) haplotype had a score of 5.6.

TABLE 3
Maize markers significantly associated with anthracnose stalk resistance (2021).
All significances and positions are as presented as in previous tables.

| | | Physical | | | | | | | SNP |
|---|---|---|---|---|---|---|---|---|---|
| | | Position | RP | Donor | | INBRED | | SEQ | Pos in |
| Marker | p-value | (B73 v5) | Allele | Allele | Het | A | PH1M6A | ID | Seq |
| PHM12 | 9.92E−03 | 2,555,169 | 4.14 | 4.41 | 3.11 | C | T | 21 | 61 |
| 19705-9 | 2.09E−03 | 2,713,132 | 4.33 | 3.13 | 4.22 | T | C | 22 | 56 |
| 19707-15 | 7.64E−12 | 2,715,911 | 4.99 | 2.96 | 2.64 | A | C | 23 | 51 |
| C01964-1 | 1.05E−25 | 2,815,521 | 5.17 | 2.23 | 1.76 | G | T | 15 | 201 |
| C01957-1 | 2.20E−22 | 2,816,414 | 5.60 | 2.78 | 2.55 | T | C | 16 | 201 |
| SBD_INBRED | 1.97E−25 | 2,989,461 | 5.17 | 2.23 | 1.76 | A | T | 24 | 51 |
| A_24 | | | | | | | | | |
| PHM10 | 4.90E−26 | 3,445,069 | 5.64 | 2.23 | 2.43 | A | G | 25 | 51 |
| SBD_INBRED | 4.96E−21 | 3,535,236 | 5.80 | 3.40 | 2.46 | A | G | 26 | 51 |
| A_109 | | | | | | | | | |
| PZE- | 1.57E−01 | 4,430,109 | 4.17 | 2.83 | 4.16 | T | G | 20 | 51 |
| 110006361 | | | | | | | | | |

Transgenic constructs were made from 5 genes in the region between PHM12 and SBD_INBREDA_109 using sequence from INBRED A (NLR 01, NLR 02, NLR 03, NLR 04 and NLR 05); two of these were prioritized for greenhouse testing (NLR 02 and NLR 04) in 2021 and all were field tested in 2021.

### Example 1. Greenhouse Testing

For controlled environment testing, plants were inoculated 21 days after greenhouse planting. Inoculations were in the leaf sheath of the 2^{nd} elongated internode with 20 ul of a spore suspension of 500,000 spores per ml. Plants were then incubated in a dew chamber (100% RH) for 48H, then transferred to the greenhouse. Ten days after inoculation, a visual score of was given based on disease development: Resistant (very little or no visible leaf sheath necrosis), susceptible (necrotic tissue covering the leaf sheath), or intermediate.

In greenhouse testing, every transgenic line that was positive for having a copy of the construct NLR 04 showed resistance to Anthracnose stalk rot, whereas lines without the construct (null) showed susceptibility (Table 4). The other gene (NLR 02) showed possible efficacy with intermediate scores for construct positive plants (1 or 2 copies) and susceptible scores for null plants.

TABLE 4
Results of controlled environment testing of
NLR 04 and NLR 02 (Key: S—susceptible, I—intermediate,
M—missing, R—resistant.)

| Plant # | Construct | ANTROT | Transgene Copies |
|---|---|---|---|
| 421271227 | Control | S | Susc control |
| 421271229 | Control | S | Susc control |
| 421271228 | Control | S | Susc control |
| 421271196 | Control | S | Susc control |
| 421271198 | Control | S | Susc control |
| 421271197 | Control | S | Susc control |
| 421270797 | Control | S | Susc control |
| 421270798 | Control | S | Susc control |
| 421270796 | Control | S | Susc control |
| 421271418 | NLR 02 | I | 2 |
| 421271403 | NLR 02 | I | 2 |
| 421271401 | NLR 02 | I | 2 |
| 421271331 | NLR 02 | I | 2 |
| 421271312 | NLR 02 | I | 2 |
| 421271309 | NLR 02 | I | 1 |
| 421271382 | NLR 02 | I | 1 |
| 421271211 | NLR 02 | I | 2 |
| 421271225 | NLR 02 | I | 2 |
| 421271218 | NLR 02 | I | 2 |
| 421271285 | NLR 02 | I | 2 |
| 421271280 | NLR 02 | I | 2 |
| 421271272 | NLR 02 | I | 2 |
| 421271233 | NLR 02 | I | 1 |
| 421271235 | NLR 02 | I | 1 |
| 421271236 | NLR 02 | I | 1 |
| 421271219 | NLR 02 | M | Null |
| 421271353 | NLR 02 | R | 1 |
| 421271404 | NLR 02 | S | Null |
| 421271415 | NLR 02 | S | Null |
| 421271406 | NLR 02 | S | Null |
| 421271307 | NLR 02 | S | Null |
| 421271311 | NLR 02 | S | Null |
| 421271310 | NLR 02 | S | Null |
| 421271373 | NLR 02 | S | Null |
| 421271368 | NLR 02 | S | Null |
| 421271370 | NLR 02 | S | Null |
| 421271206 | NLR 02 | S | Null |
| 421271212 | NLR 02 | S | Null |
| 421271259 | NLR 02 | S | Null |
| 421271254 | NLR 02 | S | Null |
| 421271263 | NLR 02 | S | Null |
| 421271237 | NLR 02 | S | Null |
| 421271238 | NLR 02 | S | Null |
| 421271242 | NLR 02 | S | Null |
| 421271260 | NLR 04 | R | 1 |
| 421271257 | NLR 04 | R | 1 |
| 421271266 | NLR 04 | R | 1 |
| 421271396 | NLR 04 | R | 1 |
| 421271397 | NLR 04 | R | 1 |
| 421271392 | NLR 04 | R | 1 |
| 421271359 | NLR 04 | R | 1 |
| 421271376 | NLR 04 | R | 1 |
| 421271367 | NLR 04 | R | 1 |
| 421271336 | NLR 04 | R | 1 |
| 421271340 | NLR 04 | R | 1 |
| 421271338 | NLR 04 | R | 1 |
| 421271268 | NLR 04 | S | Null |
| 421271262 | NLR 04 | S | Null |
| 421271264 | NLR 04 | S | Null |
| 421271394 | NLR 04 | S | Null |
| 421271395 | NLR 04 | S | Null |
| 421271399 | NLR 04 | S | Null |
| 421271365 | NLR 04 | S | Null |
| 421271369 | NLR 04 | S | Null |
| 421271371 | NLR 04 | S | Null |
| 421271342 | NLR 04 | S | Null |
| 421271343 | NLR 04 | S | Null |
| 421271345 | NLR 04 | S | Null |

### Example 2. Field Testing

For field testing, plants were inoculated with a C. graminicola spore suspension by stalk injection 10 days after flowering as described U.S. Pat. No. 10,161,009. Scoring was done 4 weeks after inoculation by removing the tops of the plants at the ear, splitting the stalks and using a visual score for ANTROT severity (ANTINODES, ANTGR75 and ANTSUM) based on the stalk area affected by lesions caused by C. graminicola. Severity was determined based on the number of internodes showing discoloration (ANTINODES), the number of internodes showing greater than 75% discoloration (ANTGR75), and the sum of these (ANTSUM). Scores of 1-3 are considered susceptible, scores between 4-6 are intermediate, and scores 7-10 are classified as resistant. One of the genes (NLR 04) was found to be clearly associated with resistance to the trait (Table 5) in plants with 1 copy of the transgenic construct. The other gene (NLR 02) which showed possible efficacy in the greenhouse, did not show efficacy in the field.

TABLE 5
Summary of field testing results of NLR
04 across 4 events and 2 locations.

| | Average of | Average of | Average of | Count of |
|---|---|---|---|---|
| Genotype | ANTINODES | ANTGR75 | ANTSUM | ANTSUM |
| Hemizygous | 1.56 | 0.68 | 2.25 | 126 |
| Undetermined | 1.25 | 0.25 | 1.50 | 4 |
| WildType | 2.76 | 1.84 | 4.61 | 157 |
| Control | 2.84 | 2.05 | 4.89 | 225 |

1. A method of obtaining a progeny maize plant comprising a marker allele associated with anthracnose stalk rot resistance, the method comprising:
a. providing a population of maize plants and isolating nucleic acids from each of the population of maize plants;
b. analyzing each of the isolated nucleic acids for the presence of a marker allele on chromosome 10 that is associated with anthracnose stalk rot resistance, wherein the marker allele comprises one or more of:
i. a “C” at PHM12 at position 61 of SEQ ID NO: 21,
ii. a “T” at 19705-9 at position 56 of SEQ ID NO: 22,
iii. an “A” at 19707-15 at position 51 of SEQ ID NO: 23,
iv. a “G” at C01964-1 at position 201 of SEQ ID NO: 15,
v. a “T” at C01957-1 at position 201 of SEQ ID NO: 16,
vi. an “A” at SBD_INBREDA 24 at position 51 of SEQ ID NO: 24,
vii. an “A” at PHM10 at position 51 of SEQ ID NO: 25, or
viii. an “A” at SBD_INBREDA 109 at position 51 of SEQ ID NO: 26;
c. selecting one or more maize plant in which the marker allele is detected;
d. crossing the selected one or more maize plants with one or more second maize plants to obtain a progeny plant comprising the marker allele.

2. The method of claim 1, wherein the method comprises analyzing each of the isolated nucleic acids for the presence of a combination of two or more of the following marker alleles:
i. a “C” at PHM12 at position 61 of SEQ ID NO: 21,
ii. a “T” at 19705-9 at position 56 of SEQ ID NO: 22,
iii. an “A” at 19707-15 at position 51 of SEQ ID NO: 23,
iv. a “G” at C01964-1 at position 201 of SEQ ID NO: 15,
v. a “T” at C01957-1 at position 201 of SEQ ID NO: 16,
vi. an “A” at SBD_INBREDA_24 at position 51 of SEQ ID NO: 24,
vii. an “A” at PHM10 at position 51 of SEQ ID NO: 25, or
viii. an “A” at SBD_INBREDA_109 at position 51 of SEQ ID NO: 26.

3. The method of claim 1, wherein the method comprises analyzing each of the nucleic acids for the presence of each of marker alleles:
i. a “C” at PHM12 at position 61 of SEQ ID NO: 21,
ii. a “T” at 19705-9 at position 56 of SEQ ID NO: 22,
iii. an “A” at 19707-15 at position 51 of SEQ ID NO: 23,
iv. a “G” at C01964-1 at position 201 of SEQ ID NO: 15,
v. a “T” at C01957-1 at position 201 of SEQ ID NO: 16,
vi. an “A” at SBD_INBREDA_24 at position 51 of SEQ ID NO: 24,
vii. an “A” at PHM10 at position 51 of SEQ ID NO: 25, and
viii. an “A” at SBD_INBREDA_109 at position 51 of SEQ ID NO: 26.

4. The method of any one of claims 1-3, further comprising planting the progeny maize plant in a field having, or at risk for having, C. graminicola.

5-9. (canceled)

10. A method of increasing resistance to Anthracnose stalk rot in a plant, comprising expressing in a plant a heterologous polynucleotide that encodes a polypeptide comprising an amino acid sequence of at least 95% sequence identity to an amino acid sequence of SEQ ID NO: 30; wherein the plant expressing the polypeptide has increased resistance to Anthracnose stalk rot in the plant when compared to a control plant not comprising the heterologous polynucleotide.

11. The method of claim 10, wherein the polypeptide encoded by the heterologous polynucleotide comprises the amino acid sequence set forth in SEQ ID NO: 30.

12-13. (canceled)

14. A recombinant DNA construct comprising a first polynucleotide operably linked to at least one regulatory sequence wherein the first polynucleotide comprises a nucleic acid sequence encoding an amino acid sequence of at least 95% sequence identity to the sequence set forth in SEQ ID NO: 30, wherein said allelic variant is associated with increased resistance to Anthracnose stalk rot,
wherein the recombinant DNA construct further comprises a second polynucleotide adjacent to the first polynucleotide, wherein said second polynucleotide comprises a nucleic acid sequence encoding a disease resistance protein comprising a sequence different from that which is set forth in SEQ ID NO: 30, and
wherein the first and second polynucleotides are not naturally adjacent in the maize genome.

15. (canceled)

16. The recombinant DNA construct of claim 14, wherein the first polynucleotide encodes a polypeptide comprising the nucleic acid sequence set forth in SEQ ID NO: 30.

17. A recombinant plant or plant cell comprising the recombinant DNA construct of claim 14, wherein the construct is heterologous to the plant or plant cell.

18. (canceled)

19. A seed produced from the plant of claim 17, wherein the seed comprises the recombinant, heterologous construct.

20. The method of claim 10, wherein the heterologous polynucleotide comprises the nucleotide sequence set forth in SEQ ID NO: 28.

21. The method of claim 10, wherein the heterologous polynucleotide comprises a molecular stack comprising a plurality of disease resistance genes.


SEQUENCE LISTING (30 sequences)

SEQ ID NO: 1 - DNA, 241 bp, Zea Maize
cgcggacgtggcgctggccagggctgggcagcctacgatcgccgtsgcsggctgcttcgctggcccsatgttcaacatgctggtcggkctrggaaccgcccttgtggtacagacggcgagggtgtatcccggagcttttgtgctcgagttccaygttgggatygtcgtcgcgtttgtgttcctgctcctgagcctgatgggcaccctgttggtggtcacctgggctaggtttcgggtgccc

SEQ ID NO: 2 - DNA, 241 bp, Zea Maize
  misc_feature (21)..(28): n is a, c, g, or t
aattttcttcactgtttgtgnnnnnnnnttactactawtcatctccagaatataaagggtagttcacatcattaacttattttggcagtgtgctgtaaacttcagctctgagaacataccaaatggtattggatttggagggcagccacatcatttcggactctttttatcagcaaattttgaccaagggcactcatttacgtgtagcacatttaccagccctcccctttccaagacaaat

SEQ ID NO: 3 - DNA, 241 bp, Zea Maize
  misc_feature (198)..(204): n is a, c, g, or t
tcataaacttccagactttgtacactactagttactataagcccactaatacccttttggtcaaacagcaggtaagcactgaagcaaaattcatcyttgatgccaattatgttatcgtcattggctgtacctttggcgcttacctggaatattgttgctccctggtgtttccaaatyccagctgctggcttggccwannnnnnnaaacgraaacctccaggctccagctgctagctgtcat

SEQ ID NO: 4 - DNA, 241 bp, Zea Maize
  misc_feature (233)..(233): n is a, c, g, or t
accacaaggccatggcagtcatgcaacatgataatatcttyaaaatcrcgaagactgaaaygcaagatagtaagcwttgtctcctgttatcactrcccagagaaataattatgagtaactcatttctaattctgtcaccaaatccttgatattcatrggytgctrgagtaggtgmtatttggtaccagacagttcagtcatgtttatatttattttgttctctgtasatgttncagagaaa

SEQ ID NO: 5 - DNA, 354 bp, Zea Maize
  misc_feature (1)..(127): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsttagtgttcgccatgatcatgggagaaggtttctgcttaacaaaggaaaaagggaaaagaakcttgtattcccatgagtaaatatcaaactgctcatggtatctatgttgttgggattgcttgtatgtgtgtayatgttcgtgtgtgtcgtgtgttgatccacagctactggtagcaagagcttctatccctgctgatggaattgcgcaatgccaatgttgacatt

SEQ ID NO: 6 - DNA, 374 bp, Zea Maize
agcagatgtcactatggtcgatggycatgacaccaaggagcagacactcccttccacctctggcatgaccgattcttcgagcggyagcgcctcaagcgagcctactttcaaggggaacccaggacgtgccctcaaagaccgaagcagccctgcrctgacygtgaaagctacctacaatggcgacaccgtccggttcaagtttstgccagcaaggggctggtaccacttgctggaagagatagcaaagaggttcaagctgacggctggagcattccagctcaaatacaaggacgatgaggacgagtgggtgatcctggcraacgaygccgacctccaggagtgcatggacgtcctggactcgatcagctcacgca

SEQ ID NO: 7 - DNA, 241 bp, Zea Maize
  misc_feature (76)..(76): n is a, c, g, or t
  misc_feature (83)..(83): n is a, c, g, or t
aattgactgctctaayttctmttgatattaycccacttgttttgtcaggttgacccattctttgtaccrcagtttnccaragncactgtttctccatacacttccatacacagttacaaaacataatacgtcagttaacttgaacaaagtctctattttgtaatatgtttcccgattgcattggtttatggtaaacagttcctccaacagcgrctatctgagtcacgtctggtgatgcagt

SEQ ID NO: 8 - DNA, 241 bp, Zea Maize
cttcacacttggtaacgctagcctacatgagaggccaggcggaggcacagctgaccatggacatggatcaatccgaaccgagctgcacttgccattttgtgcgcagtgtgagcaccatcttctgcccttctacggagtagttcacattgggtacttggacaaaggaagtggagaaggcatcatcgaccggtcgcattttcaggcactggttcrtttctatgggtgcaggcttcaggtkcaa

SEQ ID NO: 9 - DNA, 238 bp, Zea Maize
  misc_feature (47)..(97): n is a, c, g, or t
ggccgagacatcccagctgagaacagccagcacaattctgacgcagnnnnnnnnnnnnnnnnnnnnnnnnnnnnnnnnnnnnnnnnnnnnnnnnnnngcaccagtagacatccaggcgatagagcatccacatcctggcctgacgwaaagttaccgtcagccaagagcagccctggtcactgctcgtccgatmtttgcattggagctatggatgtccagcgccggtctgcgtgggggt

SEQ ID NO: 10 - DNA, 121 bp, Zea Maize
ctagctgctttactaaatgtgcagccttaacatcaatggttgggagatgatgatctccataggcttcctggctgcaacagggtacgtacgcaaccacatcagtttcttatctttttttaat

SEQ ID NO: 11 - DNA, 121 bp, Zea Maize
aagagaggctgaagaaatggtacatggaagagaacagaaggatgcaggcctcgagggggaatccttgagccggtccttcaaccaacttcaagcctggcctttgtgggttgctgattgacac

SEQ ID NO: 12 - DNA, 201 bp, Zea Maize
  misc_feature (50)..(50): n is a, c, g, or t
  misc_feature (61)..(61): n is a, c, g, or t
  misc_feature (151)..(151): n is a, c, g, or t
  misc_feature (155)..(155): n is a, c, g, or t
tcaggtatatgattcagccaagttggcaaccaggtgttagtcgggctgantgattcaaatnccatgtggaatcaagaccactgattcaggcaattctataatgcaactttgaattgatttcgctgtttttaccaaaactctgaagaaattntgancggctcaggtgatcaggaaacagacagtaaaaatttcatagatcta

SEQ ID NO: 13 - DNA, 121 bp, Zea Maize
ggcaccagagcgtttgatactttgatcggtgccagtggaatccctccactgcccactggtgggcttcgctccttgtcatgtgtgcaatttgttcacgggcatgctctcaggctgagagcct

SEQ ID NO: 14 - DNA, 121 bp, Zea Maize
acgctcccattttcaatgtattctgtaggttgtgtgtaaagagctgttctacagagagttgccagtaatggatgactcacttgctgtaaagcttcttccaggatgcactacaatagacgag

SEQ ID NO: 15 - DNA, 401 bp, Zea Maize
  misc_feature (173)..(173): n is a, c, g, or t
  misc_feature (212)..(212): n is a, c, g, or t
  misc_feature (243)..(243): n is a, c, g, or t
agaccaacagatttggggggaaaaacaacaccgcaagaacaaacaaatctcactgccattttttagatattaatttaaagagattaagagagaagaaaagaagatgacccaaacaaacaaactcgagctagtagacttacggcagggttcttcagcaccgggcagggaggagnccgggaggcggaaggtgttcagttcactgattgttagcnagcgaagccaagagcaagagctactcccctntagatttgattgagatgggagaggctcaacttttgattccctcgagcacgtataatgcagctagctgcagctttaattttctaatgccatgtactcgtttcccaagagcagatcaaaacagcagacacgaggccaggagttatttgagtttgtaacat

SEQ ID NO: 16 - DNA, 401 bp, Zea Maize
ttaggtatcttctcgatttgaggtactttgaaaaattcaaaaataatgaaaatctacaaatatagtcactggcacaacagaacgatgcaaaccttaacaccgacacaagccaggaaaatgtatatatagtagtgctcaaaacatttgctatatgtaaaagatgagatgtttgtggcgactgatcgatgttaccggtcagataatcggcaaggctccctttgcttaggtattcgaagcaaaacaacctttggcgcaggtctgcccagacaaatcttccttcgtagggtgccctcacatgttgcgtctcggatgagtaccctagacatcggactatgttcctgtgcttgacctctatgagacaagcaacctcgctttggaaattcgcctctaggacatcgatc

SEQ ID NO: 17 - DNA, 121 bp, Zea Maize
  misc_feature (15)..(15): n is a, c, g, or t
tagcagcagcgacancgcggaggtgcacatgtcagcctcaagaccgggatccgctgctgctgcgtcgtcctcgtcctcctctctcagtctcagctgcaacaagcacaacccgcaggccgcc

SEQ ID NO: 18 - DNA, 121 bp, Zea Maize
  misc_feature (51)..(51): n is a, c, g, or t
ggaccgatgatccaatattaaaatcaagtaagcagcgcaacaagtgaccantgattgtgcaccgtgcaccagtatatctaactcgactcatgtttctacgcagctggttttgtaaatacat

SEQ ID NO: 19 - DNA, 121 bp, Zea Maize
acagcagcaattggagctgtggtggttgccgggagtggcgatgattgacttctgaacttcattgcatctgttaatagtgccagctgccggctgttatcacaaggtaaacacaccgacattc

SEQ ID NO: 20 - DNA, 101 bp, Zea Maize
tgaaagtttagttttgaaatgctgtaaccgaatagagcggcaaagaatattgggaaggctgctagaactatagctgacagtggtagcaattcacgtttgaa

SEQ ID NO: 21 - DNA, 121 bp, Zea Maize; identical to SEQ ID NO: 17
  misc_feature (15)..(15): n is a, c, g, or t

SEQ ID NO: 22 - DNA, 104 bp, Zea Maize
aattgtgaaccgatgagtataacttattttctccatattctgaacaacccattacttaaacagatttgcatcctactggagacattgctggcagaggaattgag

SEQ ID NO: 23 - DNA, 96 bp, Zea Maize
accagtgtatggttctcattcactgtcgctgtccatcatttcttgtagctagatgtagctagtgctgccttttcatcctttgaccactatacgcgc

SEQ ID NO: 24 - DNA, 102 bp, Zea Maize
caaagggggaaatagcagtagagactggagattggtgatcttacacaagtaaagtaaatcatgggcgcatggagctggaatcgacaacaagctcgaggccgc

SEQ ID NO: 25 - DNA, 101 bp, Zea Maize
ctaaagcagtcttctctagagagtgactctgccaaatcatgaaggatatcatgcatgatatagtatgaataatgtcttttagaaaccaattggaagaaaga

SEQ ID NO: 26 - DNA, 102 bp, Zea Maize
catttgcaagatccgtcaagacgtcgaagtggccgaaactgccgctagcaatgtcctacggctagacccagacgacgcgtcggtttacattcttctctctaa

SEQ ID NO: 27 - DNA, 2000 bp, Zea Maize
tgagacaggcgcaggcaaggccatcttcagtgtcccagtatcttcaattggcaagacatcatctggggtcatctctggcggcccaactcgaggtcagggcgagacatgcacagaaggcacgatctcaagcttcccaactcgaatgtggggcgagacagccccagtcatcgtcgtgttcaggagcccaggtagacagtggggcgagaccggcgctggaagcctcgtctctagcgtcgggcaaggctggggcgagacaggccttgggagccatgtctcaagcggcccagttggccgaaggggcgagaccgaggcattccaggccatctcaggcggcccaatcaggttccggggtgagcccaccaacacagacgacgttccaacagtcctgtcgcaatcaatccggggactgtttatcttccccgtggagatctgtcccaccataactgtcacggtagtctctctgtcttcctggggctccttggaaaccggtgtctcttcaatgcgcaaggaaggcagcgcatctcgatcaggatctctcaccggatatgctgtttccctacccgaggtcgttgacggcggtggaaacacagcccgaggcgccagggttgcagcgccgcgtcccaacgacatctgtgggtccaggcgaagatgaaccgagcgccgcaggaacagggacggcgcgggaaacgccgcccaaggcgccaaagttgccgcaccgcgtcccgacggcagctgcgagcccaggcgaagatgaaccggtcgcctctggaacaggggcccgtttgaacgacgagactgggggtcctggtcctcgccgccgccgccatcaccatcaggtctcctgccgtacgccggaggtgggggcggtggaggccccaggtccgccgcagatagctctgacagggtagcgctcacactgatcttgtaagtgaggcatctcttctcaagtgcgtgcaatccaggctcgacgatgagaaggtccatatcacggtgtagtcttctaggatcaaagcaccaagcactgacccaaaaggctgagtaatctttcttttgcaaagtatccggatgcacgtccgaaatccagcaagagtctctcaagatgttttctgccgtagaacgaaaccaagcgtgctctgggattcctctaatctcaacctccaccataccagacatgatcgtcgaagatgcatgagtaaacctcgaccagcgcttgaacttcaaactgaaatgaggccccctatatgccttgccatcattaagcacccttgaggcagtttcttcatcaggtagaatcaagaggaaatcctctggctggacttgatgaatctgcatggcttcaatatggacctcaaaattatgtgcgacttcttctaacacttcaactcctagcacctccggcctagtcccaacaactgttacgaacagggcacggcggagaaatacctcttcacgcgtcatctcagtggtgaattccaacacacaagaaggcaatgacttcttgaatatctccatttgtcctccagacctcagcgtcgacctatggacctttctggaggcgtcagcagtatcagaggaaaccggggacatagagcctggaccagcaccagaccccaacgatcctttgccccgtttcgagcgtcgtcttttcttcttgttgcgtaggcttctttgagagtcgcctgcagcaaacatccttgaagtgtctgccttcagcgcaggctccaacggaaacgagatcttatttgatctaatttctctagaaccttccttggaatttcgaagaaggacaacataaacatcgtaagacttgttaagatggagttaattagcactagtcgaccaacaacagacaatattttccctttccacccactaactcttttcttaaatctttttatgatataattccaatcttcattactaagctttctgtgatgcattggaatacccagatatttgaaaggataatgccctttattacatccaaaaatttcagagta

SEQ ID NO: 28 - DNA, 5827 bp, Zea Maize
ttccaattctcgttctttagcagcatcataacagaaaagttggctcttatgaaaatttattttgagacccgaaagatgttcaaacgcacaaagtagtaagccaaaactgcattcaaaggaaacgtggaggcttgtttccacacagccactattacacggaacacaactgtaatcatccagtgccttgtttttcctcatcagaaggttcctggtattgagtgtatctctatcattctagaaaagtggaaccaaataggaccgaaagcgcagaggaactgctatgccttaaggactggatgcaaattggaaatgaaagagatagttttttttaggattttggtctgtactggaagctctcaatttttatggccagatgaattgttggtccaactcatgggagatctcgtcttctacattcatatcaagctcagctcctcttgcaacgtacaaccatgtctaaaaaaattaggggaggactcccaggaaagctccaaaagctttctttgtcagtgtttattcactcgaactgcataatttagagcagtcctaatccatagactagtctactccgcttgttcacctcttgttcacctctctttccatgctgcttcctttcccagctagcagtagaccctcagcagtctacggcaccttcggtctcctcaacataaaaaaacaaatcatctatcaccagcaatcagcaacaccatccggtattcctccgttccctgcccgacggatctacttccctccttgctgtgctgtgctgtgcttgttccagattctactccatacgcccttgatttattttacttcgtgtaagatctccaatctccacttttttcgcttgctgtgttagctactatttcccctttgtgttgctggatctggcgagaaagaaaaataattggtttgtgcttcattttttcattcagattcattaattttatgtgtctgcagagaaaaaagaaaaaaaaagcttcttactgaatttcatggccgacttggcgctcgccggcttaagatgggcagcatcgccgattgtcaacgagcttcttactaaagcttcagcttacctcagtgtggacatggtgcgtgagatccaacgactagaagccactgtcctgccacagttcgagctggtgattcaagcggcccagaagagcccccacaggggcatactggaggcatggctccggcgtctcaaagaagcctactatgatgccgaggacttgttggacgagcatgagtacaatgtccttgaagttgaaggcaaggccaagagcggaaaaagtctcctgctgggagagcatggaagctcctccactgcaactactgtcacgaaaccttttcatgctgccatgagcagggcgcggaacttgctacctcaaaacagaaggctaattagcaagatgaacgagctcaaagcaatcctgacagaagcccaacaacttcgagatcttcttggtttgccacatggcaataccgtcgggtggccagctgcagcatctaccagtgttcccacaaccacatcacttcccacttccaaggtttttggtcgcgacagggatcgtgatcgtatagtagattttcttctcggcaagacaacaactgctgaggcaagctcagctaagtactcgggtttggccattgttggattgggaggaatggggaagtccaccttagcacagtatgtctataatgacaaaaggatagaagaatgctttgatatcaggatgtgggtgtgcatctcacgcaaacttgatgtgcatcgtcacacaagggagattatagagtctgcaaaaaagggagagtgcccacgtgttgataatctcgatactctccagtgcaaattacgcgatatactacaagagtcacagaaattcctgcttgtcttggatgatgtttggtttgaaaaatctcataatgagacagagtgggagttattccttgctccattagtctctaaacagtcagggagcaaagttttggtgacttctcgaagtgaaacacttccggctgctatttgttgtgaacaagaacatgtcattcatttgaaaaacatggatgatactgagtttttggctctttttaaacaccatgctttctctggagcagaaatgaaagaccaactgttacgcacgaagctggaagacactgcagaggagattgctaaaaggcttggacaatgtcctttggcagcaaaagttctgggttctcgattgtgcaggaaaaaggatattgttgaatggaaagctgctctaaagcttggagatttaagtgatcccttcacatctctgttgtggagttacgagaagttagatccacgtctgcagaggtgcttcttgtattgcagcttgtttccaaaaggtcacggatatacacctgaagagttggttcacctttgggtggcagaaggatttgttggttcatgcaatttgagtaggagaacgttagaagaggttgggatggattacttcaatgatatggtctctgtatctttcttccaattggtttctcaaatgtattgtgattcgtactatgtcatgcatgatatccttcatgattttgcagagtcactctctagggaagactgctttagattagaagatgataatgtgacagaaataccatgcactgttcgacatctatctgttcatgttcaaagtatgcaaaagcataagcaaattatctgcaagctacatcatttacgcactattatctgcatcgatccgctaatggatggtccaagtgatatttttgatggcatgctacggaaccaaagaaaactgcgtgtattgtctctgtcattttacagcagcagcaagttgccagaatctattggtgagctgaagcacctccggtatttgaacctcatcaggacgttagtttctgaattgcctacatcattatgtactctctaccacttacaattactttggttaaactacatggtggagaatttgcctgacaaactatgcaatttaagaaagctacgacatctaggagcgtactcatgttacgctcatgatttcgttgatgaaatgcctatttgccaaatctcgaatataggtaagttaacgtccctacaacacatttatgtcttttctgtacagaagaagcaaggttatgagttgcgacagttgaaggacttgaatgagcttggtggcagtttaagagtgaaaaatcttgagaatgtcattggaaaggatgaagccgtagagtcgaagctatatctgaaaagtcgccttaaagagttggcacttgagtggagttccgagaatggaatggatgcaatggatattctagaaggtctgagaccaccaccccaactgagtaagctcacaatcgaaggttacagatctgatacatatcctgggtggttactagagcgatcctattttgagaatttggaaagttttgagcttagtaattgcagtttgctagaaggcctaccaccagatacagagctccttcggaattgctctaggttgcgtataaactttgttccaaatttgaaggaactatctaatcttccagcaggccttacagatttatcaattgatcgttgcccactgcttatgtttatcaccaacaatgagctaggacagcatgacttgagggaaaatataataatgaaggcagacgacctggcatctaaacttgcattgatgtgggaggtggattcaggaaaagaagttaggagagtactgtcgaaagactgttcatctctgaagctgttgatgacattgatgatggatgatgatatatcaaagcatcttcaaatcattgaaagtggtctgaaggaaagagaagataaagtatggatgaaagaaaacatcatcaaggcatggctcttttgccatgagcagaggataagattcatttatggaaggaccatggagatgccattggttctaccgtcaggactctgtgaactttctctttcttcatgcagtattacagatgaagctttagctatttgccttggtggcctcacttcactgaggaatttaaaattgaaatataatatggcattaactacacttccatcagaaaaggtgtttgagcatttgacaaagcttgacaggttggttgtaataggttgtttgtgtctcaaatcactggggggcttacgtgctgctccatctctttccttttttaaaagttgggattgtccttctttagagctagcacggggagcagaactaatgccgttgaaccttgctagcgttctcagcatccttggctgcattcttgcagctgattcgttcattaatggcttgccacatctgaaacatctttccattgatgtctgcagaagctccccatccttatcgattggccacctgacctcccttgaatcattacgtctaaatggcctccctgatctttactttgttgaaggcttgtcttccctgcaccttaagcacctaagtttagtagatgttgcaaacctcactgccaagtgcatctcacagtttcgtgtccaggaattgctcacggttagtagctctgtattgctcaaccacatgctaatggctgaagggtttacagccccaccaaatcttactcttttagattgcaaggagccgtcagtttcatttgaagaacctgcaaatctctcatccgtcaagcacctgaagttttcatgttgcgaaacagagtccctgcctagaaatctaaaatccgtctcaagtctggagagtctttctatagaacattgccccaacatagcatctttaccagatctgccgtcctccctccagcgcataactatattgaattgccccgtcttgatgaagaattgccaagaacctgatggagaaagctggccaaagatttcgcacgttcgttggaagagctttctaccaatatcgatctgacttccttagagttgccattttgaaataaatgagaaggtacaggttctactaattcatttttccagcacaattgatgagtttctcaatatttaaaacatttcatgttctaaacaggcaccttgacgtcacccctcttctcttgaagctccagagttcaggctcaagtcagaagccaatccgtcgttaatgctgccgtccccgcggttccctgtttttgccgcttgtattgctccgctatttcagttgctatatcattcattccttggttgtgcacaattgccaatatgtatttctctgacagaatgaagtaataactgtggctagggcttttgttttcatgtgcacaattgctatatcattcatgtggcaattagtataaatactccaggcaatgcaatagagaagatagttcatgttcctgcacttgtgcttgtggatggtgtggcagagaagttcaccatggaaccagttccagtaatgtgtgtgctacagtcaaaataagtcttaattctctgctacgtacagtcaaaacgtgtacagtccgagtgtttctggagattcgttgttggcggatgatgagctacatttgtacagtatggattgagctcgaggacttgttcagctccttgctctattaccagttaaaaggtgattgcttgttctctattaccaattggattgattaccagtgtgcttgctggcgtaagagatgaaaaggccttgtattttagtgcatcaaaactgaaggttttttggtggtatgctccattctgttcaatgtcctaaaccctacacacaaaatgtaaaaccttacgcgctggtttgcaccatctataaccaaagtgtatcattcattgattgcacggcgcttctgcagcacctatctgttgctgctgctgctgatgaaccgctgtgctgttcaacaacaacgtgtccgcgcg

SEQ ID NO: 29 - DNA, 1000 bp, Zea Maize
ggcgtcaaagctattctgactgagcaggctaccataaacatcgacatccatctgttgttgcaatgctgattcagaagtgttagactatgtgtgtgtgagtgtgtgtgggccggtaccactgttgggctgccggcccattagggttagggtttcatgtgtctctatatattgtagtccacctctatgcaatatagggagttcacattccaacatggtatcaaaactgggttaggttttcttcccttcccactgcagccgccgggggctctctcccacccagcagccgccaggtgcagccgtctcccacctgagccgccgccgcccccgggagactcctccctcccggggcgaccccttccattggcgcggctgtgcacgggcgcggcccctcgcagctcggcgcgggcgtggctgcccagccttggcgcggcagcccccagctccgacggcgcgcggcgcgggtgcgcgtggaagcggacgaccctcttctccctggatccgcgccgccggtggcggatccgccagcacctcccatggctgccgtgctcctacaccgagcaccacaacaacagccagcgcaggggcctcccatggccgtcgagctctctccacgtcggcgctccctgacctcctccccatgggcgcgtcccctgcaggcgagcagcggctcccatctcccagcggcctccatggccgagctcccctggagcaggcgctttccagccatggcgcgggctccatttgtctagggccgccgaagctcctccagccgcgccctccctgttccatggcgtccaaagctccctgtgctcggcgcccttcttccacctctgactgctcctctctccaccgagcgcccctccccaactctcctacacccgaaccccctgtgccgcccaggccgcgcgccctgcaagcgaccgagccgctgcccgccgagccgccccgcgcgcacaccgcgtgatgccgcgcccaggagccgcgcccagcgcccgcgcgc

SEQ ID NO: 30 - protein, 1296 aa, Zea Maize
MADLALAGLRWAASPIVNELLTKASAYLSVDMVREIQRLEATVLPQFELVIQAAQKSPHRGILEAWLRRLKEAYYDAEDLLDEHEYNVLEVEGKAKSGKSLLLGEHGSSSTATTVTKPFHAAMSRARNLLPQNRRLISKMNELKAILTEAQQLRDLLGLPHGNTVGWPAAASTSVPTTTSLPTSKVFGRDRDRDRIVDFLLGKTTTAEASSAKYSGLAIVGLGGMGKSTLAQYVYNDKRIEECFDIRMWVCISRKLDVHRHTREIIESAKKGECPRVDNLDTLQCKLRDILQESQKFLLVLDDVWFEKSHNETEWELFLAPLVSKQSGSKVLVTSRSETLPAAICCEQEHVIHLKNMDDTEFLALFKHHAFSGAEMKDQLLRTKLEDTAEEIAKRLGQCPLAAKVLGSRLCRKKDIVEWKAALKLGDLSDPFTSLLWSYEKLDPRLQRCFLYCSLFPKGHGYTPEELVHLWVAEGFVGSCNLSRRTLEEVGMDYFNDMVSVSFFQLVSQMYCDSYYVMHDILHDFAESLSREDCFRLEDDNVTEIPCTVRHLSVHVQSMQKHKQIICKLHHLRTIICIDPLMDGPSDIFDGMLRNQRKLRVLSLSFYSSSKLPESIGELKHLRYLNLIRTLVSELPTSLCTLYHLQLLWLNYMVENLPDKLCNLRKLRHLGAYSCYAHDFVDEMPICQISNIGKLTSLQHIYVFSVQKKQGYELRQLKDLNELGGSLRVKNLENVIGKDEAVESKLYLKSRLKELALEWSSENGMDAMDILEGLRPPPQLSKLTIEGYRSDTYPGWLLERSYFENLESFELSNCSLLEGLPPDTELLRNCSRLRINFVPNLKELSNLPAGLTDLSIDRCPLLMFITNNELGQHDLRENIIMKADDLASKLALMWEVDSGKEVRRVLSKDCSSLKLLMTLMMDDDISKHLQIIESGLKEREDKVWMKENIIKAWLFCHEQRIRFIYGRTMEMPLVLPSGLCELSLSSCSITDEALAICLGGLTSLRNLKLKYNMALTTLPSEKVFEHLTKLDRLVVIGCLCLKSLGGLRAAPSLSFFKSWDCPSLELARGAELMPLNLASVLSILGCILAADSFINGLPHLKHLSIDVCRSSPSLSIGHLTSLESLRLNGLPDLYFVEGLSSLHLKHLSLVDVANLTAKCISQFRVQELLTVSSSVLLNHMLMAEGFTAPPNLTLLDCKEPSVSFEEPANLSSVKHLKFSCCETESLPRNLKSVSSLESLSIEHCPNIASLPDLPSSLQRITILNCPVLMKNCQEPDGESWPKISHVRWKSFLPISI